(19) United States
(12) Patent Application Publication
(10) Pub. No.: US 2009/0082251 A1
(43) Pub. Date: Mar. 26, 2009

(54) Mitochondrial DNA variants associated with metabolic syndrome

(75) Inventors: Douglas C. Wallace, Irvine, CA (US); Ping H. Wang, Irvine, CA (US); Lee-Ming Chuang, Taipei (TW)

Correspondence Address: QUINE INTELLECTUAL PROPERTY LAW GROUP, P.C., P O BOX 458, ALAMEDA, CA, 94501, US

(73) Assignee: The Regents of the University of California, Oakland, CA (US)

(21) Appl. No.: 12/156,958

(22) Filed: Jun. 4, 2008

Related U.S. Application Data
(60) Provisional application No. 60/933,201, filed on Jun. 4, 2007.

(51) Int. Cl. A61K 38/02; C12Q 1/68; G01N 33/50; G01N 33/68

(52) U.S. Cl. 514/2; 435/6; 436/94; 436/86

(57) ABSTRACT

Provided are methods of identifying Metabolic Syndrome phenotypes for an organism or a biological sample derived therefrom which methods are based on detecting a polymorphism, haplotype, haplotype group, or haplotype subgroup in the mitochondrial genome of the organism and correlating the polymorphism or haplotype to a Metabolic Syndrome phenotype. Also provided are systems or kits for the detection of such polymorphisms or haplotypes and the correlation of the polymorphisms or haplotypes to a Metabolic Syndrome phenotype. Provided are methods of identifying a modulator of a Metabolic Syndrome phenotype and kits for the treatment of a Metabolic Syndrome phenotype.

### CROSS-REFERENCE TO RELATED APPLICATIONS

This application is related to U.S. provisional patent application U.S. Ser. No. 60/933,201 “Mitochondrial DNA Variants Associated with Metabolic Syndrome” by Wallace, Wang and Chuang, filed Jun. 4, 2007, which is incorporated in its entirety for all purposes. The present application claims priority to, and benefit of, U.S. Ser. No. 60/933,201.

### FIELD OF THE INVENTION

The present invention relates to methods of identifying one or more Metabolic Syndrome phenotypes and kits for the detection of and treatment for Metabolic Syndrome.

### BACKGROUND OF THE INVENTION

Metabolic Syndrome is a collection of health disorders or risks that increase the chance of developing heart disease, stroke, and diabetes. The condition is also known by other names, including Syndrome X, Insulin Resistance Syndrome, and Dysmetabolic Syndrome. Metabolic Syndrome can include any of a variety of underlying metabolic phenotypes, including insulin resistance and/or obesity predisposition phenotypes.

Metabolic Syndrome is often characterized by any of a number of metabolic disorders or risk factors, which are generally considered to most typify Metabolic Syndrome when more than one of these factors are present in a single individual. The factors include: central obesity (disproportionate fat tissue in and around the abdomen), atherogenic dyslipidemia (these include a family of blood fat disorders including, e.g., high triglycerides and low HDL cholesterol, that can foster plaque buildups in the vascular system, including artery walls), high blood pressure (130/85 mmHg or higher), insulin resistance or glucose intolerance (the inability to properly use insulin or blood sugar), a chronic prothrombotic state (e.g., characterized by high fibrinogen or plasminogen activator inhibitor [−1] levels in the blood), and a chronic proinflammatory state (e.g., characterized by higher than normal levels of high-sensitivity C-reactive protein in the blood). People with Metabolic Syndrome are at increased risk of coronary heart disease, other diseases related to plaque buildups in artery walls (e.g., stroke and peripheral vascular disease) and Type 2 Diabetes.

Furthermore, predisposition to obesity, Metabolic Syndrome, insulin resistance and/or the like can occur in patient populations exposed to any of a variety of environmental factors. For example, obesity predisposition can manifest itself as a simple predisposition to put on weight when exposed to a modern diet, or it can arise as a result of specific triggering events. Metabolic Syndrome is extremely common, particularly in the United States, where roughly 50 million people are thought to have the disorder. Roughly one in five Americans has Metabolic Syndrome. The number of people with Metabolic Syndrome increases with age, affecting more than 40 percent of people in their 60s and 70s. The underlying causes of Metabolic Syndrome are, in many respects, quite unclear—though certain effects of the disorder such as obesity and lack of physical activity are often causal in nature as well. Given inheritance patterns for the disorder, there also appear to be genetic factors that underlie the syndrome.

Not only is Metabolic Syndrome likely a result of several interacting genetic and environmental factors, but also the criteria for diagnosing Metabolic Syndrome are somewhat variable. Criteria considered most relevant by the “Third Report of the National Cholesterol Education Program (NCEP) Expert Panel on Detection, Evaluation, and Treatment of High Blood Cholesterol in Adults (Adult Treatment Panel III)” in the diagnosis of metabolic disorder provide one widely used current set of diagnostic criteria.

Under the NCEP criteria, Metabolic Syndrome can be clinically identified by presence of three or more of the following components in a single patient: (1) central obesity, as measured by waist circumference (women with a waist circumference greater than 35 inches; for men greater than 40 inches); (2) fasting blood triglycerides greater than or equal to 150 mg/dL; (3) blood HDL cholesterol (for women less than 50 mg/dL, for men less than 40 mg/dL); (4) blood pressure greater than or equal to 130/85 mmHg; and (5) fasting glucose greater than or equal to 110 mg/dL. Other features such as insulin resistance (e.g., increased fasting blood insulin), prothrombotic state or proinflammatory state are not generally required for clinical diagnosis, though they are certainly also indicative of Metabolic Syndrome and follow-up studies on these attributes can be used to further confirm diagnosis of Metabolic Syndrome. For example, insulin resistance, even in the absence of the NCEP criteria, is often indicative of Metabolic Syndrome.

Treatment for Metabolic Syndrome, can include a variety of clinical approaches, including weight loss and exercise (these two safest and most effective treatments are also often quite difficult to achieve in practice), and dietary changes. These dietary changes include: maintaining a diet that limits carbohydrates to 50 percent or less of total calories; eating foods defined as complex carbohydrates, such as whole grain bread (instead of white), brown rice (instead of white), sugars that are unrefined, increasing fiber consumption by eating legumes (for example, beans), whole grains, fruits and vegetables, reducing intake of red meats and poultry, consumption of “healthy” fats, such as those in olive oil, flaxseed oil and nuts, limiting alcohol intake, etc. In addition, treatment of blood pressure, and blood triglyceride levels can be controlled by a variety of available drugs (e.g., cholesterol modulating drugs), as can clotting disorders (e.g., via aspirin therapy) and in general, prothrombotic or proinflammatory states. If Metabolic Syndrome leads to diabetes, there are, of course, many treatments available for this disease, including those noted above, in conjunction with insulin treatment.

While a considerable amount is known about Metabolic Syndrome and its phenotypes, e.g., obesity, insulin resistance, and hypertension, at the clinical level, disease diagnosis for these central human diseases is relatively imprecise, and early detection of susceptible individuals is difficult. The present invention provides a number of new genetic correlations between Metabolic Syndrome (including e.g., obesity predisposition and insulin resistance), and various mitochondrial polymorphisms and haplotypes providing the basis for improved diagnosis of disease, early detection of susceptible individuals (e.g., before Metabolic Syndrome or weight gain is clinically manifested), targets for potential disease modulators, as well as an improved understanding of Metabolic Syndrome, obesity, and dyslipidemia at the molecular and cellular level. These and other features of the invention will be apparent upon review of the following.

### SUMMARY OF THE INVENTION

This invention is generally directed to identifying Metabolic Syndrome phenotypes and/or phenotypes associated with other mitochondrial diseases, e.g., Leber's Hereditary Optic Neuropathy (LHON), by detecting various mitochondrial polymorphisms, haplotype groups and/or haplotype subgroups that were previously unknown to be correlated with Metabolic Syndrome or, e.g., LHON. The detection of these polymorphisms can provide the basis for improved diagnosis of Metabolic Syndrome and/or LHON and for early detection of these disorders in susceptible individuals, e.g., individuals that have not yet exhibited symptoms. The invention also provides methods id identifying a modulator of a Metabolic Syndrome phenotype. Identification of such modulators of, e.g., an obesity predisposition, dyslipidemia, an insulin resistance phenotype, or other phenotypes of Metabolic Syndrome can provide the basis for treatments of this disease.

Accordingly, in one aspect, the invention provides two sets of methods of identifying a Metabolic Syndrome phenotype for an organism or biological sample derived from an organism, e.g., a human patient or a biological sample derived from a human patient (blood, lymph, skin, tissue, saliva, primary or secondary cell cultures derived therefrom, etc.). The first set of methods includes detecting a polymorphism, haplotype, haplotype subgroup or haplotype group in a mitochondrial genome of the organism or biological sample and correlating the polymorphism or haplotype to the Metabolic Syndrome phenotype. The second set of methods includes detecting a polymorphism, haplotype, haplotype subgroup or haplotype group noted in the tables herein in an organism or biological sample, wherein the polymorphism, haplotype, haplotype subgroup or haplotype group is associated with the Metabolic Syndrome phenotype, and correlating the polymorphism, haplotype, haplotype subgroup or haplotype group to the Metabolic Syndrome phenotype.

The correlation between a Metabolic Syndrome phenotype and a haplotype or haplotype subgroup in either set of methods can optionally comprise one or more of the following: E and waist circumference; F3 and waist circumference; F4 and increased risk for obesity, waist circumference and body mass index (BMI); M10 and decreased levels of triglycerides and systolic and diastolic blood pressure (SBP, DBP); N9a and decreased risk for type 2 diabetes (T2DM), cholesterol, and high density lipoprotein levels (HDL) in men; R9 and overall Metabolic Syndrome (MS); D and increased SBP; D5 and elevated cholesterol and SBP in women but decreased BMI for men; and D4b for very low triglycerides. Optionally, correlating the polymorphism can comprise referencing a look up table that comprises correlations between alleles of the polymorphism and the phenotype.

Detecting a polymorphism, haplotype, haplotype subgroup or haplotype group in a mitochondrial genome of the organism or biological sample in either set of methods can optionally comprise amplifying the polymorphism or a sequence associated therewith and detecting the resulting amplicon. Optionally, amplifying can comprise performing a polymerase chain reaction (PCR), reverse transcriptase PCR (RT-PCR), or ligase chain reaction (LCR) using mitochondrial nucleic acid isolated from the organism or biological sample as a template in the PCR, RT-PCR, or LCR. The amplicon can optionally be detected by a process that includes one or more of the following steps: hybridizing the amplicon to an array, digesting the amplicon with a restriction enzyme, and real-time PCR analysis. Detecting a polymorphism, haplotype, haplotype subgroup or haplotype group in a mitochondrial genome of the organism or biological sample in either set of methods can optionally comprise partially or fully sequencing the amplicon.

In a related aspect, the invention provides systems or kits that include an amplification composition or set of amplification reagents comprising one or more amplification primers that flank or comprise one or more polymorphisms that distinguish one or more haplotypes, haplotype subgroups or haplotype groups selected from: E, F3, F4, M10, N9a, R9, D, D5, and D4b. The systems or kits of the invention also include a look up table that correlates one or more of the haplotypes, haplotype subgroups, or haplotype groups to one or more metabolic syndrome phenotype. The systems or kits can optionally comprise one or more containers that contain the amplification composition or amplification primers. Optionally, the systems or kits can include computer-implemented instructions that correlate a product of the amplification composition or reagents with the Metabolic Syndrome phenotype using the look up table.

The invention also provides methods of identifying a modulator of a Metabolic Syndrome phenotype. These methods include contacting a potential modulator to a gene or gene product, wherein the gene or gene product comprises a polymorphism within, or is at least partially encoded within a haplotype selected from: E, F3, F4, M10, N9a, R9, D, D5 and D4b, and detecting an effect of the potential modulator on the gene or gene product. The effect of the potential modulator on the gene or gene product can optionally comprise increased or decreased expression of a gene encoding or corresponding to a polymorphism or haplotype herein in the presence of the modulator. The Metabolic Syndrome phenotypes for which modulators can be identified include insulin resistance, a lipid disorder, or central obesity.

Relatedly, the invention provides kits for treatment of a Metabolic Syndrome phenotype. The kits include a modulator identified by any of the methods described above, wherein the methods comprise amplifying the polymorphism or a sequence associated therewith in a mitochondrial genome and detecting the amplicon. The kits also include instructions for administering the compound to a patient to treat the Metabolic Syndrome phenotype. The Metabolic Syndrome phenotypes that are treated by the kits can optionally include an obesity predisposition, dyslipidemia, or an insulin resistance phenotype.

### DEFINITIONS

Before describing the present invention in detail, it is to be understood that this invention is not limited to particular devices or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a polymorphism” includes a combination of two or more polymorphisms or a set of polymorphisms; reference to “nucleic acid” optionally includes many copies of that nucleic acid molecule.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used in accordance with the definitions set out below.

A “phenotype” is a trait or collection of traits that is/are observable in an individual or population. The trait can be quantitative (a quantitative trait, or QTL) or qualitative.

A “Metabolic Syndrome phenotype” is a phenotype that displays a predisposition towards developing Metabolic Syndrome in an individual, or that displays Metabolic Syndrome in the individual. A phenotype that displays a predisposition for Metabolic Syndrome, can for example, show a higher likelihood that the syndrome will develop in an individual with the phenotype than in members of the general population under a given set of environmental conditions, such as a high calorie, e.g., high-fat, and/or high-carbohydrate diet, and/or a low physical activity regime. Metabolic Syndrome can be characterized by any of a number of metabolic disorders or risk factors, generally considered to most typify Metabolic Syndrome when more than one of these factors are present in a single individual. The factors include: central obesity (disproportionate fat tissue in and around the abdomen), atherogenic dyslipidemia (these include a family of blood fat disorders including, e.g., high triglycerides and low HDL cholesterol, that can foster plaque buildups in the vascular system, including artery walls), high blood pressure (e.g., 130/85 mmHg or higher), insulin resistance or glucose intolerance (the body can't properly use insulin or blood sugar), a chronic prothrombotic state (e.g., characterized by high fibrinogen or plasminogen activator inhibitor [−1] levels in the blood), and a chronic proinflammatory state (e.g., characterized by higher than normal levels of high-sensitivity C-reactive protein in the blood).

An “insulin resistance phenotype” is a phenotype that displays a predisposition for developing insulin resistance in an individual or that display insulin resistance in the individual. For example, an individual with the phenotype can show a higher likelihood that the syndrome will develop in the individual than in members of the general population under a given set of environmental conditions (e.g., those noted above for Metabolic Syndrome). Any of a variety of tests in current use can be used to determine insulin resistance, including: the Oral Glucose Tolerance Test (OGTT), Fasting Blood Glucose (FBG), Normal Glucose Tolerance (NGT), Impaired Glucose Tolerance (IGT), Impaired Fasting Glucose (IFG), Homeostasis Model Assessment (HOMA), the Quantitative Insulin Sensitivity Check Index (QUICKI) and the Intravenous Insulin Tolerance Test (IVITT). See also, De Vegt (1998) “The 1997 American Diabetes Association criteria versus the 1985 World Health Organization criteria for the diagnosis of abnormal glucose tolerance: poor agreement in the Hoorn Study.” Diab Care 1998, 21:1686-1690; Matthews (1985) “Homeostasis model assessment: insulin resistance and B-cell function from fasting plasma glucose and insulin concentrations in man.” Diabetologia 28:412-419; Katz, A (2000) “Quantitative Insulin Sensitivity Check Index: A Simple, Accurate Method for Assessing Insulin Sensitivity In Humans.” JCE & M 85:2402-2410. It will be appreciated that patients with insulin resistance can also suffer from Metabolic Syndrome and/or obesity.

An “obesity predisposition phenotype” is a phenotype that displays a predisposition for developing obesity (e.g., central obesity) in an individual, or that displays obesity. For example, an individual with the phenotype can show a higher likelihood that obesity will develop in the individual than in members of the general population under a given set of environmental conditions (e.g., those noted above for Metabolic Syndrome). “Central obesity” is a trait characterized by a large and/or disproportionate deposit of fat around the waist. Most women with a waist of greater than 35 inches, and most men with a waist of greater than 40 inches are classified as having central obesity. It will be appreciated that patients with Metabolic Syndrome are often obese, and/or insulin resistant; the three phenotypes are all interrelated.

A “polymorphism” is a locus that is variable; that is, within a population, the nucleotide sequence at a polymorphism has more than one version. One example of a polymorphism is a “single nucleotide polymorphism” (SNP), which is a polymorphism at a single nucleotide position in a genome (the nucleotide at the specified position varies between individuals or populations).

The term “haplotype” refers to a set of nucleic acid polymorphisms, e.g., SNPs, on, e.g., a mitochondrial DNA, that are inherited as a unit. A “haplotype group” is a group of similar haplotypes that share a common ancestor.

A marker polymorphism or haplotype is “correlated” with a specified phenotype (Metabolic Syndrome, LHON, etc.) when it can be statistically linked (positively or negatively) to the phenotype. This correlation is often inferred as being causal in nature, but it need not be—simple genetic linkage to (association with) a locus for a trait that underlies the phenotype is sufficient.

A polymorphism or haplotype “positively” correlates with a trait when it is linked to it and when presence of the polymorphism or haplotype is an indictor that the trait or trait form will occur in an individual comprising the polymorphism or haplotype. A polymorphism or haplotype negatively correlates with a trait when it is linked to it and when presence of the polymorphism or haplotype is an indicator that a trait or trait form will not occur in an individual comprising the polymorphism or haplotype.

A “marker,” “molecular marker” or “marker nucleic acid” refers to a nucleotide sequence used as a point of reference when identifying a locus or a linked locus. A marker can be derived from, e.g., an mtDNA nucleotide sequence. The term also refers to nucleic acid sequences complementary to or flanking the marker sequences, such as nucleic acids used as probes or primer pairs capable of amplifying the marker sequence. A “marker probe” is a nucleic acid sequence or molecule that can be used to identify the presence of a marker locus, e.g., a nucleic acid probe that is complementary to a marker locus sequence. Nucleic acids are “complementary” when they specifically hybridize in solution, e.g., according to Watson-Crick base pairing rules. A “marker locus” is a locus that can be used to track the presence of a second linked locus, e.g., a linked or correlated locus that encodes or contributes to the population variation of a phenotypic trait. In one aspect, the present invention provides marker loci correlating with a phenotype of interest, e.g., /Metabolic Syndrome, LHON, etc. Each of the identified markers is expected to be in close or overlapping physical and genetic proximity (resulting in physical and/or genetic linkage) to a genetic element, e.g., a QTL, that contributes to the relevant phenotype. Alternately, the marker locus can be in linkage disequilibrium (LD) with the marker.

The term “amplifying” in the context of nucleic acid amplification is any process whereby additional copies of a selected nucleic acid (or a transcribed form thereof) are produced. Typical amplification methods include various polymerase based replication methods, including the polymerase chain reaction (PCR), ligase mediated methods such as the ligase chain reaction (LCR) and RNA polymerase based amplification (e.g., by transcription) methods. An “amplicon” is an amplified nucleic acid, e.g., a nucleic acid that is produced by amplifying a template nucleic acid by any available amplification method (e.g., PCR, LCR, transcription, or the like).

A “look up table” is a table that correlates one form of data to another, or one or more forms of data with a predicted outcome to which the data is relevant. For example, a look up table can include a correlation between allele data and a predicted trait that an individual comprising one or more given alleles is likely to display. These tables can be, and typically are, multidimensional, e.g., taking multiple alleles into account simultaneously, and, optionally, taking other factors into account as well, such as genetic background, e.g., in making a trait prediction.

A “computer readable medium” is an information storage media that can be accessed by a computer using an available or custom interface. Examples include memory (e.g., ROM or RAM, flash memory, etc.), optical storage media (e.g., CD-ROM), magnetic storage media (computer hard drives, floppy disks, etc.), punch cards, and many others that are commercially available. Information can be transmitted between a system of interest and the computer, or to or from the computer or to or from the computer readable medium for storage or access of stored information. This transmission can be an electrical transmission, or can be made by other available methods, such as an IR link, a wireless connection, or the like.

“System instructions” are instruction sets that can be partially or fully executed by the system. Typically, the instruction sets are present as system software.

An “array” is an assemblage of elements. The assemblage can be spatially ordered (a “patterned array”) or disordered (a “randomly patterned” array). The array can form or comprise one or more functional elements (e.g., a probe region on a microarray) or it can be non-functional.

A “locus” is a position or region on, e.g., an mtDNA. For example, a polymorphic locus is a position or region where a polymorphic nucleic acid, trait determinant, gene or marker is located.

### BRIEF DESCRIPTION OF THE DRAWINGS

FIG. 1 depicts standard peak distributions for a healthy individual.

FIG. 2 depicts calibration curves of mtDNA extension peak heights.

FIG. 3 depicts the correlation of expected and observed percentages of 3243 A→G heteroplasmy.

FIG. 4 depicts the results of the direct extension of mtDNA.

FIG. 5 depicts the output of the primer generation program used in the example.

FIG. 6 provides a schematic depiction of a method for diagnosing a mitochondrial disease.

FIG. 7 depicts the organization of a global SNP haplotyping strategy.

FIG. 8 provides standard multiplex haplotyping panels.

FIG. 9 provides histograms. Panel A provides a histogram that depicts a correlation between body mass index and F4 haplogroups vs. non-F4 haplogroups. Panel B provides a histogram that depicts a correlation between waist circumference and F4 haplogroups vs. non-F4 haplogroups. Panel C provides a histogram that depicts a correlation between triglycerides and M10 haplogroups vs. non-M10 haplogroups. Panel D provides a histogram that depicts a correlation between systolic blood pressure and M10 haplogroups vs. non-M10 haplogroups. Panel E provides a histogram that depicts a correlation between diastolic blood pressure and M10 haplogroups vs. non-M10 haplogroups. Panel F provides a histogram that depicts a correlation between fasting glucose and N9a haplogroups vs. non-N9a haplogroups. Panel G provides a histogram that depicts a correlation between HDL-C and N9a haplogroups vs. non-N9a haplogroups. Panel H provides a histogram that depicts a correlation between HDL-C and men with N9a haplogroups vs. men with non-N9a haplogroups. Panel I provides a histogram that depicts a correlation between fasting glucose and men with N9a haplogroups vs. men with non-N9a haplogroups. Panel J provides a histogram that depicts a correlation between total cholesterol and men with N9a haplogroups vs. men with non-N9a haplogroups. Panel K provides a histogram that depicts a correlation between total cholesterol and D5 haplogroups vs. non-D5 haplogroups. Panel L provides a histogram that depicts a correlation between triglycerides and D4b haplogroups vs. non-D4b haplogroups. Panel M provides a histogram that depicts a correlation between total cholesterol and women with D5 haplogroups vs. women with non-D5 haplogroups. Panel N provides a histogram that depicts a correlation between systolic blood pressure and women with D5 haplogroups vs. women with non-D5 haplogroups. Panel O provides a histogram that depicts a correlation between body mass index and men with D5 haplogroups vs. men with non-D5 haplogroups. Panel P provides a histogram that depicts a correlation between total cholesterol and D5 haplogroups vs. non-D5 haplogroups. Panel Q provides a histogram that depicts a correlation between triglycerides and D4b haplogroups vs. non-D4b haplogroups.

### DETAILED DESCRIPTION

### Overview

The invention includes new correlations between certain mtDNA polymorphisms, haplotypes, haplotype groups and/or haplotype subgroups and symptoms of Metabolic Syndrome, e.g., Type 2 Diabetes, hypertension, and abnormal blood lipid chemistry. Specific associations that have been discovered include: E for waist circumference; F3 for waist circumference; F4 for increased risk for obesity, waist circumference, and body mass index (BMI); M10 for decreased levels of triglycerides and systolic and diastolic blood pressure (SBP, DBP); N9a for decreased risk for type 2 diabetes (T2DM), cholesterol, and high density lipoprotein levels (HDL), particularly in men; R9 for overall Metabolic Syndrome (MS); D for increased SBP; D5 for elevated cholesterol and SBP in women but decreased BMI for men; and D4b for very low triglycerides. Accordingly, detection of these mtDNA polymorphisms, haplotypes, haplotype groups, and/or haplotype subgroups, by any available method, can be used for diagnostic purposes such as early detection of susceptibility to Metabolic Syndrome, prognosis for patients that present with the Metabolic Syndrome, and in assisting diagnosis, e.g., where current criteria are insufficient for a definitive diagnosis. (The tables and figures included elsewhere herein provide further information regarding these associations). The present invention also shows that detection of other mtDNA polymorphisms and haplotypes can be useful in diagnosing other mitochondrial diseases, e.g., Leber's Hereditary Optic Neuropathy (LHON).

The identification that mtDNA polymorphisms, haplotypes, and/or haplotype subgroups, e.g., E, F3, F4, M10, N9a, R9, D, D5, and D4b, are correlated to the symptoms of Metabolic Syndrome noted above also provides a platform for screening potential modulators of metabolic disorders. Modulators of the activity of a gene or gene product, wherein the gene or gene product corresponds to a polymorphism or haplotype from a table herein, are expected to have an effect the symptoms of Metabolic Syndrome, e.g., an obesity phenotype, dyslipidemia, and/or others. Thus, methods of screening, systems for screening and the like, are features of the invention. Modulators identified by these screening approaches are also a feature of the invention.

Kits for the diagnosis and treatment of Metabolic Syndrome, e.g., comprising reagents to identify relevant mitochondrial polymorphisms and/or haplotypes, packaging materials, and instructions for correlating detection of relevant alleles to metabolic diseases are also a feature of the invention. These kits can also include modulators of Metabolic Syndrome and/or instructions for treating patients using conventional methods.

### Methods of Identifying Metabolic Syndrome Phenotypes

As noted, the invention provides the discovery that certain mitochondrial polymorphisms, haplotypes, and/or sub-haplotypes, e.g., E, F3, F4, M10, N9a, R9, D, D5, and D4b, are linked to Metabolic Syndrome phenotypes, e.g., hypertension, and dyslipidemia. Thus, by detecting markers, e.g., the mitochondrial haplotypes, polymorphisms, or loci closely linked thereto, that correlate, positively or negatively, with the relevant phenotypes, it can be determined whether an individual or population is likely to be susceptible to these phenotypes, to Metabolic Syndrome, and/or to other mitochondrial diseases, e.g., LHON. This provides enhanced early detection options to identify patients that are likely to eventually suffer from these phenotypes, making it possible, in some cases, to prevent actual development of Metabolic Syndrome, obesity, diabetes, LHON, etc., e.g., by taking early preventative action (e.g., any existing therapy such as diet, exercise, available medications, etc.). In addition, use of the various mtDNA markers herein also adds certainty to existing diagnostic techniques for identifying whether a patient is suffering from, e.g., Metabolic Syndrome, which can be somewhat ambiguous using previous methods, e.g., as discussed in the Background of the Invention, above. Furthermore, knowledge of whether there is a molecular basis for central obesity, Metabolic Syndrome, LHON, etc., can also assist in determining patient prognosis, e.g., by providing an indication of how likely it is that a patient can respond to conventional therapy for the relevant disorder, or whether more serious options such as gastric surgery are likely to be necessary. Disease treatment can also be targeted based on what type of molecular disorder the patient displays.

Detection methods for detecting relevant alleles can include any available method, e.g., amplification technologies. For example, detection can include amplifying a relevant mitochondrial haplotype, sub-haplotype, polymorphism, or a sequence associated therewith and detecting the resulting amplicon. This can include admixing an amplification primer or amplification primer pair with, e.g., a mitochondrial nucleic acid template isolated from the organism or biological sample (e.g., comprising the SNP or other polymorphism), e.g., where the primer or primer pair is complementary or partially complementary to at least a portion of the haplotype or tightly linked polymorphism, or to a sequence proximal thereto. The primer is typically capable of initiating nucleic acid polymerization by a polymerase on the nucleic acid template. The primer or primer pair is extended, e.g., in a DNA polymerization reaction (PCR, etc.) comprising a polymerase and the template nucleic acid, e.g., an mtDNA, to generate the amplicon. The amplicon is detected by any available detection process, e.g., sequencing, hybridizing the amplicon to an array (or affixing the amplicon to an array and hybridizing probes to it), digesting the amplicon with a restriction enzyme (e.g., RFLP), real-time PCR analysis, single nucleotide extension, allele-specific hybridization, or the like.

The correlation between a detected polymorphism and a trait can be performed by any method that can identify a relationship between a polymorphism and/or haplotype and a phenotype. Most typically, these methods involve referencing a look up table that comprises correlations between, e.g., the mitochondrial haplotype, the haplotype subgroup, and/or the polymorphism, and the phenotype. The table can include data for multiple polymorphism- and/or haplotype-phenotype relationships and can take account of additive or other higher order effects of multiple polymorphism- and/or haplotype-phenotype relationships, e.g., through the use of statistical tools such as principle component analysis, heuristic algorithms, etc.

Within the context of these methods, the following discussion focuses on marker detection methods. Additional sections below discuss data analysis.

Marker Amplification Strategies

Amplification primers for amplifying markers (e.g., mitochondrial polymorphisms, mitochondrial haplotypes and/or mitochondrial haplotype subgroups) and suitable probes for detecting such markers or for genotyping a sample with respect to multiple markers, are a feature of the invention. Indeed, it will be appreciated that amplification is not a requirement for marker detection—for example, one can directly detect unamplified mitochondrial DNA simply by performing a Southern blot on a sample of mitochondrial DNA. Procedures for performing Southern blotting, standard amplification (PCR, LCR, or the like) and many other nucleic acid detection methods are well established and are taught, e.g., in Sambrook et al., Molecular Cloning—A Laboratory Manual (3rd Ed.), Vol. 1-3, Cold Spring Harbor Laboratory, Cold Spring Harbor, N.Y., 2008 (“Sambrook”); Current Protocols in Molecular Biology, F. M. Ausubel et al., eds., Current Protocols, a joint venture between Greene Publishing Associates, Inc. and John Wiley & Sons, Inc., (supplemented through 2008) (“Ausubel”)) and PCR Protocols A Guide to Methods and Applications (Innis et al. eds) Academic Press Inc. San Diego, Calif. (1997) (Innis).

Separate detection probes can also be omitted in amplification/detection methods, e.g., by performing a real time amplification reaction that detects product formation by modification of the relevant amplification primer upon incorporation into a product, incorporation of labeled nucleotides into an amplicon, or by monitoring changes in molecular rotation properties of amplicons as compared to unamplified precursors (e.g., by fluorescence polarization).

Typically, molecular markers are detected by any established method available in the art, including, without limitation, allele specific hybridization (ASH), detection of single nucleotide extension, array hybridization (optionally including ASH), or other methods for detecting single nucleotide polymorphisms (SNPs), amplified fragment length polymorphism (AFLP) detection, amplified variable sequence detection, randomly amplified polymorphic DNA (RAPD) detection, restriction fragment length polymorphism (RFLP) detection, self-sustained sequence replication detection, simple sequence repeat (SSR) detection, single-strand conformation polymorphisms (SSCP) detection, isozyme marker detection, quantitative amplification of mRNA or cDNA, or the like.

### EXAMPLE TECHNIQUES FOR MARKER DETECTION

The invention provides molecular markers, e.g., mtDNA polymorphisms, haplotypes, and/or haplotype subgroups, that are correlated with Metabolic Syndrome phenotypes. Similar methods apply to detection of correlations between LHON and molecular markers. The markers find use in disease predisposition diagnosis, prognosis, and treatment. It is not intended that the invention be limited to any particular method for the detection of these markers.

Markers corresponding to genetic polymorphisms between members of a population can be detected by numerous methods well-established in the art (e.g., PCR-based sequence specific amplification, restriction fragment length polymorphisms (RFLPs), isozyme markers, allele specific hybridization (ASH), array based hybridization, amplified variable sequences of the genome, self-sustained sequence replication, simple sequence repeat (SSR), single nucleotide polymorphism (SNP), random amplified polymorphic DNA (“RAPD”) or amplified fragment length polymorphisms (AFLP). In one additional embodiment, the presence or absence of a molecular marker is determined simply through nucleotide sequencing of the polymorphic marker region. Any of these methods are readily adapted to high throughput analysis.

Some techniques for detecting genetic markers utilize hybridization of a probe nucleic acid to nucleic acids corresponding to the genetic marker (e.g., amplified nucleic acids produced using mtDNA as a template). Hybridization formats, including, but not limited to: solution phase, solid phase, mixed phase, or in situ hybridization assays are useful for allele detection. An extensive guide to the hybridization of nucleic acids is found in Tijssen (1993) Laboratory Techniques in Biochemistry and Molecular Biology—Hybridization with Nucleic Acid Probes Elsevier, New York, as well as in Sambrook, Berger and Ausubel.

For example, markers that comprise restriction fragment length polymorphisms (RFLP) are detected, e.g., by hybridizing a probe which is typically a sub-fragment (or a synthetic oligonucleotide corresponding to a sub-fragment) of the nucleic acid to be detected to restriction digested mtDNA. The restriction enzyme is selected to provide restriction fragments of at least two alternative (or polymorphic) lengths in different individuals or populations. Determining one or more restriction enzyme that produces informative fragments for each allele of a marker is a simple procedure, well known in the art. After separation by length in an appropriate matrix (e.g., agarose or polyacrylamide) and transfer to a membrane (e.g., nitrocellulose, nylon, etc.), the labeled probe is hybridized under conditions which result in equilibrium binding of the probe to the target followed by removal of excess probe by washing.

Nucleic acid probes to the mitochondrial marker loci can be cloned and/or synthesized. Any suitable label can be used with a probe of the invention. Detectable labels suitable for use with nucleic acid probes include, for example, any composition detectable by spectroscopic, radioisotopic, photochemical, biochemical, immunochemical, electrical, optical or chemical means. Useful labels include biotin for staining with labeled streptavidin conjugate, magnetic beads, fluorescent dyes, radiolabels, enzymes, and calorimetric labels. Other labels include ligands that bind to antibodies labeled with fluorophores, chemiluminescent agents, and enzymes. A probe can also constitute radiolabelled PCR primers that are used to generate a radiolabelled amplicon. Labeling strategies for labeling nucleic acids and corresponding detection strategies can be found, e.g., in Haugland (2003) Handbook of Fluorescent Probes and Research Chemicals Ninth Edition by Molecular Probes, Inc. (Eugene Oreg.). Additional details regarding marker detection strategies are found below.

Amplification-Based Detection Methods

PCR and LCR are in particularly broad use as amplification and amplification-detection methods for amplifying nucleic acids of interest (e.g., mtDNA), facilitating detection of polymorphisms and/or haplotypes of interest. Details regarding the use of these and other amplification methods can be found in any of a variety of standard texts, including, e.g., Sambrook, Ausubel, and Berger. Many available biology texts also have extended discussions regarding PCR and related amplification methods. See also, Ausubel, Sambrook and Berger, above.

Real Time Amplification/Detection Methods

In one aspect, real time PCR or LCR is performed on the amplification mixtures described herein, e.g., using molecular beacons or TaqMan™ probes. A molecular beacon (MB) is an oligonucleotide or PNA which, under appropriate hybridization conditions, self-hybridizes to form a stem and loop structure. The MB has a label and a quencher at the termini of the oligonucleotide or PNA; thus, under conditions that permit intra-molecular hybridization, the label is typically quenched (or at least altered in its fluorescence) by the quencher. Under conditions where the MB does not display intra-molecular hybridization (e.g., when bound to a target nucleic acid, e.g., to a region of an amplicon during amplification), the MB label is unquenched. Details regarding standard methods of making and using MBs are well established in the literature and MBs are available from a number of commercial reagent sources. See also, e.g., Leone et al. (1995) “Molecular beacon probes combined with amplification by NASBA enable homogenous real-time detection of RNA.” Nucleic Acids Res. 26:2150-2155; Tyagi and Kramer (1996) “Molecular beacons: probes that fluoresce upon hybridization” Nature Biotechnology 14:303-308; Blok and Kramer (1997) “Amplifiable hybridization probes containing a molecular switch” Mol Cell Probes 11:187-194; Hsuih et al. (1997) “Novel, ligation-dependent PCR assay for detection of hepatitis C in serum” J Clin Microbiol 34:501-507; Kostrikis et al. (1998) “Molecular beacons: spectral genotyping of human alleles” Science 279:1228-1229; Sokol et al. (1998) “Real time detection of DNA:RNA hybridization in living cells” Proc. Natl. Acad. Sci. U.S.A. 95:11538-11543; Tyagi et al. (1998) “Multicolor molecular beacons for allele discrimination” Nature Biotechnology 16:49-53; Bonnet et al. (1999) “Thermodynamic basis of the chemical specificity of structured DNA probes” Proc. Natl. Acad. Sci. U.S.A. 96:6171-6176; Fang et al. (1999) “Designing a novel molecular beacon for surface-immobilized DNA hybridization studies” J. Am. Chem. Soc. 121:2921-2922; Marras et al. (1999) “Multiplex detection of single-nucleotide variation using molecular beacons” Genet. Anal. Biomol. Eng. 14:151-156; and Vet et al. (1999) “Multiplex detection of four pathogenic retroviruses using molecular beacons” Proc. Natl. Acad. Sci. U.S.A. 96:6394-6399. Additional details regarding MB construction and use is found in the patent literature, e.g., U.S. Pat. No. 5,925,517 (Jul. 20, 1999) to Tyagi et al. entitled “Detectably labeled dual conformation oligonucleotide probes, assays and kits;” U.S. Pat. No. 6,150,097 to Tyagi et al (Nov. 21, 2000) entitled “Nucleic acid detection probes having non-FRET fluorescence quenching and kits and assays including such probes” and U.S. Pat. No. 6,037,130 to Tyagi et al (Mar. 14, 2000), entitled “Wavelength-shifting probes and primers and their use in assays and kits.”

PCR detection and quantification using dual-labeled fluorogenic oligonucleotide probes, commonly referred to as “TaqMan™” probes, can also be performed according to the present invention. These probes are composed of short (e.g., 20-25 base) oligodeoxynucleotides that are labeled with two different fluorescent dyes. On the 5′ terminus of each probe is a reporter dye, and on the 3′ terminus of each probe a quenching dye is found. The oligonucleotide probe sequence is complementary to an internal target sequence present in a PCR amplicon. When the probe is intact, energy transfer occurs between the two fluorophores and emission from the reporter is quenched by the quencher by FRET. During the extension phase of PCR, the probe is cleaved by 5′ nuclease activity of the polymerase used in the reaction, thereby releasing the reporter from the oligonucleotide-quencher and producing an increase in reporter emission intensity. Accordingly, TaqMan™ probes are oligonucleotides that have a label and a quencher, where the label is released during amplification by the exonuclease action of the polymerase used in amplification. This provides a real time measure of amplification during synthesis. A variety of TaqMan™ reagents are commercially available, e.g., from Applied Biosystems (Division Headquarters in Foster City, Calif.) as well as from a variety of specialty vendors such as Biosearch Technologies (e.g., black hole quencher probes). Further details regarding dual-label probe strategies can be found, e.g., in WO92/02638.

Other similar methods include, e.g., fluorescence resonance energy transfer between two adjacently hybridized probes, e.g., using the “LightCycler®” format described in U.S. Pat. No. 6,174,670.

Array-Based Marker Detection

Array-based detection can be performed using commercially available arrays, e.g., from Affymetrix (Santa Clara, Calif.) or other manufacturers. Reviews regarding the operation of nucleic acid arrays include Sapolsky et al. (1999) “High-throughput polymorphism screening and genotyping with high-density oligonucleotide arrays.” Genetic Analysis: Biomolecular Engineering 14:187-192; Lockhart (1998) “Mutant yeast on drugs” Nature Medicine 4:1235-1236; Fodor (1997) “Genes, Chips and the Human Genome.” FASEB Journal 11:A879; Fodor (1997) “Massively Parallel Genomics.” Science 277: 393-395; and Chee et al. (1996) “Accessing Genetic Information with High-Density DNA Arrays.” Science 274: 610-614.

Additional Details Regarding Types of Markers Appropriate for Screening

The biological markers that can be screened for correlation to the Metabolic Syndrome phenotypes herein and for LHON include mtDNA polymorphisms and/or mtDNA haplotypes, haplotype groups, and haplotype subgroups, e.g., the mtDNA markers described herein. The nucleic acid of interest, e.g., mtDNA, that is to be amplified and/or detected in the methods of the invention is most preferably derived from human sources, e.g., because of its relevance to the detection of markers associated with disease diagnosis and clinical applications. The sequences for many mitochondrial haplotypes are available, including for E, F3, F4, M10, N9a, R9, D, D5, and D4b. Common sequence repositories for known nucleic acids include GenBank® EMBL, DDBJ and the NCBI. Sequence repositories for mitochondrial DNA include MitoMap, MitoSearch, and EMPOP. Other repositories can easily be identified by searching the Internet. Any variation in a mtDNA sequence can be detected as a marker, e.g., a mutation, a polymorphism, a single nucleotide polymorphism (SNP), an allele, etc. One can detect variation in sequence as markers that can be correlated to a Metabolic Syndrome phenotype or to an LHON phenotype.

For example, the methods of the invention are useful in screening samples derived from patients for a mtDNA polymorphism or an mtDNA haplotype group or haplotype subgroup, e.g., from bodily fluids (blood, saliva, urine etc.), tissue, and/or waste from the patient. Thus, stool, sputum, saliva, blood, lymph, tears, sweat, urine, vaginal secretions, ejaculatory fluid or the like can easily be screened for nucleic acids by the methods of the invention, as can essentially any tissue of interest that contains the appropriate nucleic acids. These samples are typically taken, following informed consent, from a patient by standard medical laboratory methods.

Prior to amplification and/or detection of an mtDNA marker of interest, e.g., any one or more of the markers described herein, the nucleic acid is optionally purified from the samples by any available method, e.g., those taught in Berger and Kimmel, Guide to Molecular Cloning Techniques, Methods in Enzymology volume 152 Academic Press, Inc., San Diego, Calif. (Berger); Sambrook et al., Molecular Cloning—A Laboratory Manual (3rd Ed.), Vol. 1-3, Cold Spring Harbor Laboratory, Cold Spring Harbor, N.Y., 2008 (“Sambrook”); and/or Current Protocols in Molecular Biology, F. M. Ausubel et al., eds., Current Protocols, a joint venture between Greene Publishing Associates, Inc. and John Wiley & Sons, Inc., (supplemented through 2008) (“Ausubel”)). Useful methods are also described in, e.g., Leister and Herrmann, Mitochondria: Practical Protocols Humana Press, 2007; and Leister and Herrmann, Mitochondrial Genomics and Proteomics Protocols Human Press, 2007. A plethora of kits are also commercially available for the purification of mtDNA cells or other samples (see, e.g., Wizard™ MiniPreps DNA Purification System, QIAprep™ Micro Kit from Qiagen; and kits from PromoKine and BioVision). Alternately, samples can simply be directly subjected to amplification or detection, e.g., following aliquotting and/or dilution.

Probe/Primer Synthesis Methods

In general, synthetic methods for making oligonucleotides, including probes, primers, molecular beacons, PNAs, LNAs (locked nucleic acids), etc., are well known. For example, oligonucleotides can be synthesized chemically according to the solid phase phosphoramidite triester method described by Beaucage and Caruthers (1981), Tetrahedron Letts., 22(20):1859-1862, e.g., using a commercially available automated synthesizer, e.g., as described in Needham-VanDevanter et al. (1984) Nucleic Acids Res., 12:6159-6168. Oligonucleotides, including modified oligonucleotides can also be ordered from a variety of commercial sources known to persons of skill. There are many commercial providers of oligo synthesis services, and thus this is a broadly accessible technology. Any nucleic acid can be custom ordered from any of a variety of commercial sources, such as The Midland Certified Reagent Company (mcrc@oligos.com), The Great American Gene Company (www.genco.com), ExpressGen Inc. (www.expressgen.com), Operon Technologies Inc. (Alameda, Calif.) and many others. Similarly, PNAs can be custom ordered from any of a variety of sources, such as PeptidoGenic (pkim@ccnet.com), HTI Bio-products, inc. (htibio.com), BMA Biomedicals Ltd (U.K.), Bio•Synthesis, Inc., and many others.

Amplification Primers for Marker Detection

In some preferred embodiments, the molecular markers of the invention are detected using a suitable PCR-based detection method, where the size or sequence of the PCR amplicon is indicative of the absence or presence of the marker (e.g., a particular marker allele). In these types of methods, PCR primers are hybridized to the conserved regions flanking the polymorphic marker region. Suitable primers to be used with the invention can be designed using any suitable method. It is not intended that the invention be limited to any particular primer or primer pair. For example, primers can be designed using any suitable software program, such as LASERGENE®, e.g., taking account of publicly available sequence information.

In some embodiments, the primers of the invention are radiolabelled, or labeled by any suitable means (e.g., using a non-radioactive fluorescent tag), to allow for rapid visualization of the different size amplicons following an amplification reaction without any additional labeling step or visualization step. In some embodiments, the primers are not labeled, and the amplicons are visualized following their size resolution, e.g., following agarose or acrylamide gel electrophoresis. In some embodiments, ethidium bromide staining of the PCR amplicons following size resolution allows visualization of the different size amplicons.

It is not intended that the primers of the invention be limited to generating an amplicon of any particular size. For example, the primers used to amplify the marker loci and alleles herein are not limited to amplifying the entire region of the relevant locus. In some embodiments, marker amplification produces an amplicon at least 20 nucleotides in length, or alternatively, at least 50 nucleotides in length, or alternatively, at least 100 nucleotides in length, or alternatively, at least 200 nucleotides in length.

### Correlating Markers to Phenotypes

One aspect of the invention is a description of correlations between mitochondrial polymorphisms and/or mitochondrial haplotypes, e.g., E, F3, F4, M10, N9a, R9, D, D5, and D4b, and Metabolic Syndrome phenotypes. Correlations between mitochondrial polymorphisms and/or haplotypes and LHON are also provided in the tables and figures herein. An understanding of these correlations can be used in the present invention to correlate information regarding a set of polymorphisms that an individual or sample is determined to possess and a phenotype that they are likely to display. Further, higher order correlations that account for combinations of alleles in one or more different genes can also be assessed for correlations to phenotype.

These correlations can be performed by any method that can identify a relationship between an mtDNA polymorphism and/or and mtDNA haplotype group/subgroup and a phenotype, or a combination of mtDNA polymorphisms and a combination of phenotypes. For example, polymorphisms in one or more of E, F3, F4, M10, N9a, R9, D, D5, and D4b can be correlated with one or more Metabolic Syndrome phenotype. Most typically, these methods involve referencing a look up table that comprises correlations between alleles of the polymorphism and the phenotype. The table can include data for multiple polymorphism- or haplotype-phenotype relationships and can take account of additive or other higher order effects of multiple polymorphism-phenotype relationships, e.g., through the use of statistical tools such as principle component analysis, heuristic algorithms, etc.

Correlation of a marker to a phenotype optionally includes performing one or more statistical tests for correlation. Many statistical tests are known, and most are computer-implemented for ease of analysis. A variety of statistical methods of determining associations/correlations between phenotypic traits and biological markers are known and can be applied to the present invention. For an introduction to the topic, see, Hartl (1981) A Primer of Population Genetics Washington University, Saint Louis Sinauer Associates, Inc. Sunderland, Mass. ISBN: 0-087893-271-2. A variety of appropriate statistical models are described in Lynch and Walsh (1998) Genetics and Analysis of Quantitative Traits, Sinauer Associates, Inc. Sunderland Mass. ISBN 0-87893-481-2. These models can, for example, provide for correlations between genotypic and phenotypic values, characterize the influence of a locus on a phenotype, sort out the relationship between environment and genotype, determine dominance or penetrance of genes, determine maternal and other epigenetic effects, determine principle components in an analysis (via principle component analysis, or “PCA”), and the like. The references cited in these texts provide considerable further detail on statistical models for correlating markers and phenotype.

Additional references that are useful in understanding data analysis applications for using and establishing correlations, principle components of an analysis, neural network modeling and the like, include, e.g., Hinchliffe, Modeling Molecular Structures, John Wiley and Sons (1996), Gibas and Jambeck, Bioinformatics Computer Skills, O'Reilly (2001), Pevzner, Computational Molecular Biology and Algorithmic Approach, The MIT Press (2000), Durbin et al., Biological Sequence Analysis: Probabilistic Models of Proteins and Nucleic Acids, Cambridge University Press (1998), and Rashidi and Buehler, Bioinformatic Basics: Applications in Biological Science and Medicine, CRC Press LLC (2000).

In any case, the marker(s), e.g., mtDNA polymorphisms and/or haplotype groups, can be used for any of a variety of genetic analyses. For example, once markers have been identified, as in the present case, they can be used in a number of different assays for detecting phenotype predispositions or for further association studies. For example, probes can be designed for microarrays that interrogate these markers. Other exemplary assays include, e.g., the TaqMan™ assays and molecular beacon assays described supra, as well as conventional PCR and/or sequencing techniques.

### Methods of Identifying Modulators of Metabolic Syndrome Phenotypes

In addition to providing various diagnostic and prognostic markers for identifying a Metabolic Syndrome phenotype and/or other mitochondrial diseases, e.g., LHON, the invention also provides methods of identifying modulators of Metabolic Syndrome phenotypes, e.g., an insulin resistance phenotype, or an obesity predisposition phenotype, hypertension, dyslipidemia, and/or others. In the methods, a potential modulator is contacted to a gene or gene product corresponding to, e.g., a polymorphism or a haplotype from a table herein. An effect of the potential modulator on the gene or gene product is detected, thereby identifying whether the potential modulator modulates the underlying molecular basis for the Metabolic Syndrome phenotype, e.g., any one or more of the phenotypes described herein.

In addition, the methods can include, e.g., administering one or more putative modulator to an individual that displays a relevant phenotype and determining whether the putative modulator modulates the phenotype in the individual, e.g., in the context of a clinical trial or treatment. This, in turn, determines whether the putative modulator is clinically useful.

Effects of interest that can be screened for include increased or decreased expression of a gene or gene product corresponding to, e.g., a polymorphism or a haplotype from a table herein, in the presence of the modulator. The precise format of the modulator screen will, of course, vary, depending on the effect(s) being detected and the equipment available. Northern analysis, quantitative RT-PCR and/or array-based detection formats can be used to distinguish expression levels of genes or gene products noted above. Protein expression levels can also be detected using available methods, such as western blotting, ELISA analysis, antibody hybridization, BIAcore, or the like. Any of these methods can be used to distinguish changes in expression levels of a gene or gene product corresponding to, e.g., a polymorphism or a haplotype from a table herein that result from a potential modulator.

Accordingly, one may screen for potential modulators of a gene or gene product corresponding to, e.g., a polymorphism or a haplotype from a table herein, for activity or expression. For example, potential modulators (small molecules, organic molecules, inorganic molecules, proteins, hormones, transcription factors, or the like) can be contacted to a cell comprising a mitochondrial polymorphism haplotype group, or haplotype subgroup of interest and an effect on activity or expression (or both) a gene or gene product corresponding to, e.g., a polymorphism or a haplotype from a table herein, can be detected, e.g., via northern analysis or quantitative (optionally real time) RT-PCR, before and after application of potential expression modulators. The assays can be performed in a high-throughput fashion, e.g., using automated fluid handling and/or detection systems, in serial or parallel fashion. Similarly, activity modulators can be tested by contacting a potential modulator to an appropriate cell using any of the activity detection methods herein, regardless of whether the activity that is detected is the result of activity modulation, expression modulation or both. These assays can be in vitro, cell-based, or can be screens for modulator activity performed on laboratory animals such as knock-out transgenic mice comprising a gene of interest.

In general, methods and sensors for detecting protein expression level and activity are available, including those taught in the various references above, including R. Scopes, Protein Purification, Springer-Verlag, N.Y. (1982); Deutscher, Methods in Enzymology Vol. 182: Guide to Protein Purification, Academic Press, Inc. N.Y. (1990); Sandana (1997) Bioseparation of Proteins, Academic Press, Inc.; Bollag et al. (1996) Protein Methods, 2^{nd} Edition Wiley-Liss, NY; Walker (1996) The Protein Protocols Handbook Humana Press, NJ, Harris and Angal (1990) Protein Purification Applications: A Practical Approach IRL Press at Oxford, Oxford, England; Harris and Angal Protein Purification Methods: A Practical Approach IRL Press at Oxford, Oxford, England; Scopes (1993) Protein Purification: Principles and Practice 3^{rd} Edition Springer Verlag, NY; Janson and Ryden (1998) Protein Purification: Principles, High Resolution Methods and Applications, Second Edition Wiley-VCH, NY; and Walker (1998) Protein Protocols on CD-ROM Humana Press, NJ; and Satinder Ahuja ed., Handbook of Bioseparations, Academic Press (2000). “Proteomic” detection methods, which detect many proteins simultaneously have been described and are also noted above, including various multidimensional electrophoresis methods (e.g., 2-d gel electrophoresis), mass spectrometry based methods (e.g., SELDI, MALDI, electrospray, etc.), or surface plasmon resonance methods. These can also be used to track protein activity and/or expression level.

Similarly, nucleic acid expression levels (e.g., mRNA) can be detected using any available method, including northern analysis, quantitative RT-PCR, or the like. References sufficient to guide one of skill through these methods are readily available, including Ausubel, Sambrook and Berger.

Potential modulator libraries to be screened for effects the expression or activity of, e.g., a gene or gene product corresponding to, e.g., a polymorphism or a haplotype from a table herein, are available. These libraries can be random, or can be targeted.

Targeted libraries include those designed using any form of a rational design technique that selects scaffolds or building blocks to generate combinatorial libraries. These techniques include a number of methods for the design and combinatorial synthesis of target-focused libraries, including morphing with bioisosteric transformations, analysis of target-specific privileged structures, and the like. In general, where information regarding structure of a gene or gene product corresponding to, e.g., a polymorphism or a haplotype from a table herein is available, likely binding partners can be designed, e.g., using flexible docking approaches, or the like. Similarly, random libraries exist for a variety of basic chemical scaffolds. In either case, many thousands of scaffolds and building blocks for chemical libraries are available, including those with polypeptide, nucleic acid, carbohydrate, and other backbones. Commercially available libraries and library design services include those offered by Chemical Diversity (San Diego, Calif.), Affymetrix (Santa Clara, Calif.), Sigma (St. Louis Mo.), ChemBridge Research Laboratories (San Diego, Calif.), TimTec (Newark, Del.), Nuevolution A/S (Copenhagen, Denmark) and many others.

Kits for treatment of a Metabolic Syndrome or LHON phenotype can include a modulator identified as noted above and instructions for administering the compound to a patient to treat, Metabolic Syndrome, or LHON, as appropriate.

The following table provides correlations between demographic data and clinical characteristics of study cohort.

TABLE 1
Demographic data and clinical characteristics of study cohort.

| | MALE | FEMALE | |
|---|---|---|---|
| | SUBJECTS | SUBJECTS | TOTAL |
| NUMBER | 219 | 269 | 488 |
| Age (year) | 53.21 ± 13.32 | 51.98 ± 14.62 | 52.50 ± 14.05 |
| BMI (kg/m²) | 26.28 ± 7.06 | 28.36 ± 7.83 | 27.51 ± 7.54 |
| WAIST CIRCUMFERENCE (CM) | 93.91 ± 17.5 | 95.88 ± 18.96 | 94.99 ± 18.32 |
| Systolic blood pressure (mmHg) | 128.4 ± 21.93 | 129.5 ± 21.4 | 128.93 ± 21.65 |
| Diastolic blood pressure (mmHg) | 78.04 ± 11.40 | 77.78 ± 11.22 | 77.91 ± 11.27 |
| HDL-CHOLESTEROL (MG/DL) | 42.82 ± 14.17 | 52.57 ± 13.96 | 48.33 ± 14.84 |
| Triglyceride (mg/dL) | 144.36 ± 106.9 | 143.6 ± 105.3 | 143.9 ± 105.9 |
| Fasting glucose (mg/dL) | 130.1 ± 48.01 | 132.2 ± 52.22 | 131.3 ± 50.33 |
| Metabolic syndrome by IDF 2005 (%) | 30.89% | 57.22% | 45.54% |
| Metabolic syndrome by AHA/NHLBI (%) | 50.00% | 63.80% | 57.72% |

The following table provides a correlation between mitochondrial haplogroup frequencies in the Taiwanese population.

TABLE 2
Mitochondrial haplogroups frequencies in the Taiwanese population

| | A | B | D2 | D5 | D4a | D4b | D | D* | E | F1 | F2 | F3 | F4 |
|---|---|---|---|---|---|---|---|---|---|---|---|---|---|
| NUMBER | 17 | 109 | 11 | 36 | 23 | 12 | 4 | 12 | 8 | 52 | 14 | 7 | 10 |
| Frequency | 3.52 | 22.34 | 2.25 | 7.37 | 4.71 | 2.46 | 0.8 | 2.46 | 1.6 | 10.67 | 2.87 | 1.43 | 2.05 |
| (%) | | | | | | | | | | | | | |

| | G | M7 | M7b | M8 | M10 | M21 | M* | N9a | N* | R9 | R11 | R* | Total |
|---|---|---|---|---|---|---|---|---|---|---|---|---|---|
| Number | 11 | 17 | 50 | 35 | 9 | 1 | 14 | 18 | 1 | 13 | 3 | 1 | 488 |
| Frequency | 2.28 | 3.52 | 10.3 | 7.24 | 1.86 | 0.2 | 2.86 | 3.73 | 0.2 | 2.66 | 0.6 | 0.2 | 100 |
| (%) | | | | | | | | | | | | | |

D refers to 1053, 3055, 1031, & 1038
N* refers to 1094
R* refers to 1098
M* refers to 1017, 1033, 1055, 1079, 1096, 1097, 2050, 1057, 3044, 3092, DMK-15, DMK-40, DMK-66, &DMK-70

The following table provides correlations between phenotypes and mitochondrial haplotypes. Corrections were made for multiple comparisons.

TABLE 3
Overview of associations with phenotypes and correction for multiple
comparisons

| | N | A | B | D2 | D5 | D4a | D4b | D | D* | E | F1 | F2 | F3 | F4 |
|---|---|---|---|---|---|---|---|---|---|---|---|---|---|---|
| AGE | 487 | 0.88 | 0.50 | 0.03 | 0.31 | 0.33 | 0.92 | — | 0.18 | 0.09 | 0.79 | 0.94 | 0.15 | 0.58 |
| SEX | 487 | 0.86 | 0.19 | 1.0 | 0.68 | 0.12 | 0.11 | 1.0 | 0.39 | 0.08 | 0.44 | 0.75 | 0.47 | 0.37 |
| T2DM | 487 | 0.65 | 0.79 | 0.23 | 0.10 | 0.44 | 0.78 | 0.13 | 0.24 | 0.73 | 0.79 | 0.42 | 0.13 | 0.76 |
| Obesity | 473 | 0.77 | 0.20 | 0.73 | 0.51 | 0.19 | 0.74 | 1.0 | 0.49 | 0.21 | 0.86 | 0.64 | 0.21 | 0.007 |
| HT | 386 | 0.07 | 0.71 | 1.0 | 0.88 | 0.07 | 0.54 | 1.0 | 0.50 | 0.48 | 0.86 | 0.96 | 1.0 | 0.16 |
| AHA MS | 350 | 0.85 | 0.62 | 1.0 | 0.07 | 0.52 | 0.17 | 0.19 | 1.0 | 0.70 | 0.18 | 0.21 | 0.58 | 0.25 |
| IDF MS | 336 | 0.93 | 0.28 | 1.0 | 0.08 | 0.12 | 0.35 | 0.50 | 1.0 | 0.69 | 0.29 | 0.09 | 0.38 | 0.18 |
| AC | 485 | 0.96 | 0.90 | 0.46 | 0.13 | 0.47 | 0.90 | 0.17 | 0.23 | 0.20 | 0.76 | 0.23 | 0.46 | 0.62 |
| PC | 253 | 0.62 | 0.42 | 0.09 | 0.09 | 0.81 | 0.89 | 0.05 | 0.10 | 0.99 | 0.47 | 0.15 | — | 0.18 |
| TG | 481 | 0.23 | 0.44 | 0.21 | 0.12 | 0.15 | 0.0047 | 0.07 | 0.91 | 0.86 | 0.27 | 0.63 | 0.16 | 0.19 |
| T-chol | 480 | 0.37 | 0.63 | 0.45 | 0.0018 | 0.18 | 0.84 | 0.31 | 0.97 | 0.36 | 0.95 | 0.28 | 0.70 | 0.81 |
| HDL-C | 301 | 0.97 | 0.93 | 0.82 | 0.95 | 0.41 | 0.23 | 0.10 | 0.83 | 0.24 | 0.64 | 0.69 | 0.43 | 0.50 |
| LDL-C | 134 | 0.61 | 0.60 | 0.09 | 0.31 | 0.10 | 0.30 | — | 0.61 | 0.82 | 0.90 | 0.86 | 0.60 | 0.50 |
| Waist | 374 | 0.34 | 0.98 | 0.93 | 0.68 | 0.90 | 0.52 | 0.70 | 0.50 | 0.014 | 0.48 | 0.21 | 0.03 | 0.0056 |
| BMI | 473 | 0.96 | 0.84 | 0.98 | 0.12 | 1.0 | 0.39 | 0.36 | 0.76 | 0.09 | 0.41 | 0.41 | 0.15 | 0.0028 |
| SBP | 327 | 0.51 | 0.23 | 0.59 | 0.06 | 0.23 | 0.25 | 0.74 | 0.12 | 0.85 | 0.20 | 0.22 | 0.99 | 0.09 |
| DBP | 327 | 0.11 | 0.57 | 0.84 | 0.42 | 0.49 | 0.71 | 0.17 | 0.05 | 0.78 | 0.73 | 0.75 | 0.53 | 0.07 |

| | G | M7 | M7b | M8 | M10 | M21 | M* | N9a | N* | R9 | R11 | R* | Overall |
|---|---|---|---|---|---|---|---|---|---|---|---|---|---|
| AGE | 0.70 | 0.11 | 0.50 | 0.99 | 0.69 | — | 0.24 | 0.53 | — | 0.09 | 0.82 | — | 0.39 |
| SEX | 1.0 | 0.24 | 0.18 | 0.93 | 0.74 | — | 1.0 | 0.66 | — | 0.93 | 1.0 | — | 0.50 |
| T2DM | 0.24 | 1.0 | 0.61 | 0.91 | 0.51 | — | 0.78 | 0.25 | — | 0.74 | 1.0 | — | 0.51 |
| Obesity | 0.73 | 0.38 | 0.59 | 0.12 | 1.0 | — | 1.0 | 0.56 | — | 1.0 | 1.0 | — | 0.17 |
| HT | 0.15 | 0.11 | 0.59 | 0.51 | 0.48 | — | 0.37 | 0.47 | — | 1.0 | 0.52 | — | 0.38 |
| AHA MS | 0.70 | 0.18 | 0.65 | 0.92 | 0.07 | — | 0.18 | 0.55 | — | 0.02 | 0.51 | — | 0.09 |
| IDF MS | 1.0 | 0.52 | 0.79 | 0.62 | 0.08 | — | 0.19 | 0.96 | — | 0.48 | 1.0 | — | 0.24 |
| AC | 0.64 | 0.80 | 0.48 | 0.59 | 0.14 | — | 0.36 | 0.046 | — | 0.94 | 0.61 | — | 0.62 |
| PC | 0.18 | 0.83 | 0.72 | 0.94 | 0.94 | — | 0.40 | 0.76 | — | 0.35 | — | — | 0.56 |
| TG | 0.53 | 0.66 | 0.11 | 0.13 | 0.015 | — | 0.67 | 0.49 | — | 0.11 | 0.69 | — | 0.03 |
| T-chol | 0.74 | 0.53 | 0.67 | 0.44 | 0.40 | — | 0.49 | 0.04 | — | 0.24 | 0.15 | — | 0.07 |
| HDL-C | 0.27 | 0.96 | 0.87 | 0.81 | 0.22 | — | 0.24 | 0.04 | — | 0.85 | 0.38 | — | 0.72 |
| LDL-C | 0.58 | 0.26 | 0.82 | 0.51 | 0.61 | — | 0.30 | 0.65 | — | 0.61 | 0.73 | — | 0.83 |
| Waist | 0.46 | 0.53 | 0.43 | 0.42 | 0.67 | — | 0.29 | 0.58 | — | 0.22 | 0.44 | — | 0.07 |
| BMI | 0.81 | 0.69 | 0.19 | 0.47 | 0.68 | — | 0.46 | 0.13 | — | 0.91 | 0.91 | — | 0.11 |
| SBP | 0.73 | 0.39 | 0.79 | 0.14 | 0.042 | — | 0.13 | 0.76 | — | 0.014 | 0.71 | — | 0.23 |
| DBP | 0.59 | 0.88 | 0.76 | 0.82 | 0.011 | — | 0.049 | 0.97 | — | 0.14 | 0.58 | — | 0.50 |

*P-values are listed for comparison between haplogroup X and other haplogroups (two groups)
** Overall P-values are P-values for multiple comparisons (25 groups)

The following tables provide correlations between phenotypes and individual mitochondrial haplogroups.

TABLE 4
Summary of individual haplogroup

| | F4 | NON-F4 | P-VALUE | |
|---|---|---|---|---|
| | HAPLOGROUPS | HAPLOGROUPS | (ADJUSTED P*) | OR (95% C.I.) |
| NUMBER | 10 | 478 | | |
| Age | 50.40 ± 11.57 | 52.52 ± 14.11 | 0.69 | |
| Sex (M/F) | 6/4 | 213/265 | 0.33 | |
| Obesity (BMI > 30 kg/m²) | 6/3 | 105/359 | [symbol] | 5.11 (0.42-18.46) |
| Waist (n = 7/367) | 114.83 ± 16.94 | 94.61 ± 18.16 | [symbol] | Invalid value* |
| BMI (n = 10/463) | 34.62 ± 7.98 | 27.35 ± 7.46 | [symbol] | See obesity |

*OR = infinite;
P = 0.10

Haplogroup M10

| | M10 | NON-M10 | | |
|---|---|---|---|---|
| | HAPLOGROUPS | HAPLOGROUPS | P-VALUE | OR (95% C.I.) |
| NUMBER | 9 | 479 | | |
| Age | 50.67 ± 13.52 | 52.51 ± 14.08 | 0.69 | |
| Sex (M/F) | 5/4 | 214/265 | 0.52 | |
| Triglyceride (n = 9/472) | 88.78 ± 50.56 | 145.01 ± 106.42 | 0.015 | 0.24 (0.03-1.91) |
| SBP (n = 7/320) | 113.29 ± 15.97 | 129.28 ± 21.65 | 0.042 | 0.27 (0.05-1.35) |
| DBP (n = 7/320) | 68.86 ± 8.47 | 78.11 ± 11.28 | 0.011 | 0.39 (0.08-1.97) |

Haplogroup N9a

| | | | P-value | |
|---|---|---|---|---|
| | N9a | Non-N9a | (adjusted P*) | OR (95% C.I.) |
| AGE | 50.44 ± 16.12 | 52.56 ± 13.99 | 0.53 | |
| SEX (M/F) | 9/9 | 210/260 | 0.66 | |
| TYPE 2 DIABETES | 6/12 | 221/248 | 0.33 (0.31) | 0.56 (0.21-1.52) |
| Obesity (BMI > 30 kg/m2) | 5/12 | 106/350 | 0.56 (0.92) | 1.38 (0.47-3.99) |
| Hypertension | 7/7 | 150/222 | 0.58 (0.45) | 1.48 (0.51-4.31) |
| AHA MS | 7/7 | 195/141 | 0.51 (0.57) | 0.72 (0.25-2.11) |
| IDF MS | 6/7 | 147/176 | 1.03 (0.96) | 1.03 (0.34-3.12) |
| Fasting glucose | 114.0 ± 43.17 | 131.0 ± 50.50 | 0.046 | 0.53 (0.20-1.36) |
| (n = 18/467) | | | | |
| Postmeal | 149.5 ± 62.07 | 164.0 ± 75.00 | 0.79 | 0.75 (0.24-2.35) |
| glucose (n = 10/243) | | | | |
| Triglyceride (n = 17/464) | 121.47 ± 55.61 | 144.78 ± 107.24 | 0.49 | 0.80 (0.28-2.31) |
| Total cholesterol | 170.0 ± 37.82 | 192.23 ± 42.33 | 0.040 | 0.34 (0.10-1.20) |
| (n = 17/273) | | | | |
| HDL-C (n = 11/290) | 40.21 ± 11.80 | 48.63 ± 14.87 | 0.040 | 2.00 (0.58-7.01) |
| LDL-C (n = 4/130) | 112.0 ± 33.25 | 120.41 ± 34.20 | 0.65 | 1.96 (0.27-14.35) |
| Waist (n = 16/358) | 99.62 ± 24.78 | 94.78 ± 18.00 | 0.58 | 0.84 (0.30-2.37) |
| BMI (n = 17/464) | 30.56 ± 10.55 | 27.39 ± 7.39 | 0.13 | See obesity |
| SBP (n = 6/321) | 130.67 ± 15.87 | 128.9 ± 21.76 | 0.76 | 3.40 (0.71-16.23) |
| DBP (n = 6/321) | 77.17 ± 6.43 | 77.93 ± 11.37 | 0.97 | 4.98 (1.04-23.79)* |

*P = 0.040

| | Women with | Women | P-value | |
|---|---|---|---|---|
| | N9a | without N9a | (adjusted P) | OR (95% C.I.) |
| Age (year) | 46.67 ± 17.60 | 52.12 ± 14.52 | 0.27 | |
| Type 2 Diabetes | 4/5 | 126/133 | 0.80 (0.98) | 0.85 (0.22 ± 3.21) |
| Obesity | 4/5 | 74/175 | 0.34 (0.69) | 1.89 (0.49-7.25) |
| Hypertension | 4/2 | 76/114 | 0.19 (0.16) | 3.00 (0.54-16.79) |
| AHA MS | 5/2 | 120/69 | 0.67 (0.72) | 1.44 (0.27-7.61) |
| IDF MS | 5/2 | 102/78 | 0.44 (0.49) | 1.91 (0.36-10.11) |
| Fasting glucose | 132.355.9 | 132.1 ± 52.2 | 0.95 | 0.80 (0.20-3.28) |
| (n = 9/258) | | | | |
| Postprandial glucose | 180.8 ± 65.8 | 168.4 ± 76.7 | 0.38 | 2.50 (0.30-2.12) |
| (n = 4/130) | | | | |
| TG (n = 8/258) | 120.8 ± 53.24 | 144.4 ± 106.4 | 0.78 | 0.68 (0.13-3.43) |
| Total Chol (n = 8/257) | 184.5 ± 30.10 | 195.3 ± 44.78 | 0.67 | 0.46 (0.09-2.32) |
| HDL-C (n = 6/164) | 47.72 ± 6.09 | 52.75 ± 14.14 | 0.51 | 1.08 (0.21-5.49) |
| LDL-C (n = 3/71) | 123.3 ± 29.8 | 121.4 ± 32.74 | 0.85 | 3.07 (0.27-35.49) |
| Waist (9/201) | 104.1 ± 22.89 | 95.51 ± 18.75 | 0.21 | 1.75 (0.21-14.40) |
| BMI (9/249) | 32.22 ± 10.52 | 28.22 ± 7.70 | 0.22 | See obesity |
| SBP (2/160) | 148.0 ± 2.83 | 129.26 ± 21.44 | 0.19 | Invalid value* |
| DBP (2/160) | 80.0 ± 0.00 | 77.75 ± 11.29 | 0.53 | Invalid value** |

*OR = infinite; P = 0.14
**OR = infinite; P = 0.04

| | | Men | P-value | |
|---|---|---|---|---|
| | Men with N9a | without N9a | (adjusted P) | OR (95% C.I.) |
| Age (year) | 54.22 ± 14.45 | 53.16 ± 13.30 | 0.82 | |
| Type 2 Diabetes | 2/7 | 94/115 | 0.18 (0.19) | 0.35 (0.07-1.72) |
| Obesity | 1/7 | 32/175 | 0.82 (0.74) | 0.78 (0.09-6.57) |
| Hypertension | 3/5 | 74/108 | 0.86 (0.82) | 0.88 (0.20-3.78) |
| AHA MS | 2/5 | 75/72 | 0.25 (0.27) | 0.38 (0.07-2.04) |
| IDF MS | 1/5 | 45/98 | 0.44 (0.42) | 0.44 (0.05-3.84) |
| Fasting glucose | 95.67 ± 8.51 | 131.7 ± 48.46 | [symbol] | 0.36 (0.09-1.39) |
| (n = 9/209) | | | | |
| Postprandial glucose | 128.7 ± 55.07 | 158.9 ± 74.24 | 0.30 | 0.27 (0.05-1.54) |
| (n = 6/113) | | | | |
| Triglyceride (n = 9/206) | 122.1 ± 60.86 | 145.3 ± 108.5 | 0.45 | 0.89 (0.22-3.67) |
| Total Chol (n = 9/126) | 157.11 ± 40.89 | 188.44 ± 38.86 | [symbol] | 0.24 (0.03-1.94) |
| HDL-C (n = 5/126) | 31.2 ± 10.76 | 43.28 ± 14.13 | 0.02 | 5.00 (0.54-4.60) |
| LDL-C (n = 1/59) | 78 | 119.22 ± 36.14 | 0.22 | Invalid value* |
| Waist (n = 7/156) | 93.91 ± 27.71 | 93.91 ± 17.04 | 0.40 | 0.46 (0.09-2.42) |
| BMI (n = 8/207) | 28.70 ± 10.97 | 26.39 ± 6.89 | 0.32 | See obesity |
| SBP (n = 4/161) | 122.0 ± 10.80 | 128.54 ± 22.13 | 0.64 | 1.89 (0.34-10.58) |
| DBP (n = 4/161) | 75.75 ± 7.86 | 78.10 ± 11.48 | 0.66 | 2.48 (0.44-13.90) |

*OR = 0

D5

| | D5 | Non-D5 | P-value | OR (95% C.I.) |
|---|---|---|---|---|
| AGE | 54.64 ± 12.96 | 52.31 ± 14.14 | 0.31 | |
| SEX (M/F) | 15/21 | 205/248 | 0.68 | |
| Triglyceride (n = 34/447) | 170.82 ± 119.21 | 141.92 ± 104.68 | 0.12 | 2.04 (1.01-4.11) |
| Total cholesterol | 215.11 ± 47.28 | 189.64 ± 41.45 | [symbol] | Invalid value* |
| (n = 34/446) | | | | |
| HDL-C (n = 26/275) | 49.13 ± 19.70 | 48.25 ± 14.34 | 0.95 | 1.51 (0.67-3.42) |
| LDL-C (n = 15/119) | 128.97 ± 38.32 | 119.05 ± 33.53 | 0.31 | 0.94 (0.30-2.93) |
| SBP (n = 26/301) | 137.54 ± 27.33 | 128.19 ± 20.98 | 0.06 (0.047) | 1.35 (0.60-3.06) |
| DBP (n = 26/301) | 79.07 ± 12.97 | 77.81 ± 11.15 | 0.42 | 1.43 (0.64-3.19) |

*OR = 0

D4b

| | D4b | Non-D4b | P-value | OR (95% C.I.) |
|---|---|---|---|---|
| AGE | 52.03 ± 15.31 | 52.49 ± 14.04 | 0.92 | |
| SEX (M/F) | 8/4 | 212/265 | 0.11 | |
| Triglyceride | 83.03 ± 25.64 | 145.52 ± 106.72 | [symbol] | Invalid value* |
| (n = 12/469) | | | | |
| Total cholesterol | 187.25 ± 40.43 | 191.56 ± 42.43 | 0.84 | Invalid value* |
| (n = 12/468) | | | | |
| HDL-C (n = 7/294) | 55.6 ± 20.47 | 48.15 ± 14.68 | 0.23 | 0.42 (0.08-2.20) |
| LDL-C (n = 3/131) | 100.37 ± 28.89 | 120.62 ± 34.15 | 0.30 | Invalid value* |

*OR = 0

The following tables provide correlations between individual phenotypes and mitochondrial haplogroups.

Triglyceride

| | A | B | D2 | D5 | D4a | D4b | D | D* |
|---|---|---|---|---|---|---|---|---|
| NUMBER | 17 | 108 | 11 | 34 | 22 | 12 | 4 | 12 |
| VALUE | 109.11 ± 50.30 | 155.92 ± 137.98 | 166.3 ± 87.79 | 170.8 ± 119.2 | 112.82 ± 50.08 | 83.08 ± 25.64 | 78.0 ± 37.89 | 136.08 ± 78.20 |

| | E | F1 | F2 | F3 | F4 | G | M7 | M7b |
|---|---|---|---|---|---|---|---|---|
| NUMBER | 8 | 51 | 14 | 7 | 9 | 11 | 17 | 50 |
| VALUE | 154.13 ± 110.5 | 123.8 ± 65.27 | 120.14 ± 55.75 | 92.71 ± 44.32 | 189.4 ± 125.77 | 156.9 ± 103.5 | 118.9 ± 50.35 | 162.4 ± 107.9 |

| | M8 | M10 | M21 | M* | N9a | N* | R9 | R11 | R* | P |
|---|---|---|---|---|---|---|---|---|---|---|
| NUMBER | 35 | 9 | 1 | 14 | 17 | 1 | 13 | 3 | 1 | |
| VALUE | 162.9 ± 116.9 | 88.77 ± 50.56 | 133 | 142.3 ± 106.3 | 121.4 ± 55.61 | 131 | 207.2 ± 181.1 | 149.7 ± 77.93 | 70 | 0.03 |

Waist circumference

| | A | B | D2 | D5 | D4a | D4b | D | D* |
|---|---|---|---|---|---|---|---|---|
| NUMBER | 16 | 72 | 9 | 24 | 20 | 11 | 3 | 10 |
| VALUE | 88.00 ± 12.52 | 95.05 ± 18.54 | 97.76 ± 20.85 | 96.50 ± 19.00 | 98.21 ± 22.39 | 92.78 ± 19.67 | 92.47 ± 2.73 | 98.22 ± 19.27 |

| | E | F1 | F2 | F3 | F4 | G | M7 | M7b |
|---|---|---|---|---|---|---|---|---|
| NUMBER | 6 | 40 | 12 | 6 | 7 | 10 | 12 | 40 |
| VALUE | 79.27 ± 12.84 | 92.95 ± 17.23 | 97.05 ± 12.68 | 80.33 ± 9.07 | 114.83 ± 16.93 | 99.24 ± 21.42 | 91.62 ± 16.76 | 96.85 ± 19.04 |

| | M8 | M10 | M21 | M* | N9a | N* | R9 | R11 | R* | P |
|---|---|---|---|---|---|---|---|---|---|---|
| NUMBER | 29 | 8 | 1 | 10 | 16 | 1 | 10 | 2 | — | |
| VALUE | 97.53 ± 19.18 | 95.78 ± 14.77 | 102.4 | 88.52 ± 12.63 | 99.62 ± 24.77 | 27.53 | 87.66 ± 8.49 | 85.5 ± 0.71 | — | 0.07 |

Body mass index

| | A | B | D2 | D5 | D4a | D4b | D | D* | E |
|---|---|---|---|---|---|---|---|---|---|
| NUMBER | 17 | 106 | 11 | 32 | 23 | 12 | 4 | 12 | 7 |
| VALUE | 26.07 ± 5.09 | 27.31 ± 7.66 | 28.71 ± 9.74 | 25.87 ± 6.67 | 28.26 ± 8.81 | 27.05 ± 10.99 | 28.46 ± 5.42 | 28.73 ± 8.27 | 23.22 ± 2.66 |

| | F1 | F2 | F3 | F4 | G | M7 | M7b | M8 | M10 | M21 |
|---|---|---|---|---|---|---|---|---|---|---|
| NUMBER | 49 | 14 | 7 | 10 | 11 | 16 | 49 | 35 | 9 | 1 |
| VALUE | 26.85 ± 7.00 | 28.88 ± 7.46 | 23.44 ± 3.17 | 34.62 ± 7.98 | 28.93 ± 9.86 | 26.57 ± 6.53 | 27.80 ± 6.58 | 28.86 ± 8.34 | 26.53 ± 7.15 | 26.99 |

| | M* | N9a | N* | R9 | R11 | R* | P |
|---|---|---|---|---|---|---|---|
| NUMBER | 14 | 17 | 1 | 13 | 2 | 1 | |
| VALUE | 25.57 ± 5.21 | 30.56 ± 10.55 | 27.53 | 26.28 ± 4.66 | 21.95 ± 2.51 | 22.77 | 0.11 |

Total cholesterol

| | A | B | D2 | D5 | D4a | D4b | D | D* | E |
|---|---|---|---|---|---|---|---|---|---|
| NUMBER | 17 | 109 | 10 | 34 | 21 | 12 | 4 | 12 | 8 |
| VALUE | 187.2 ± 53.3 | 189.8 ± 43.2 | 173.5 ± 24.3 | 215.1 ± 47.3 | 178.5 ± 30.6 | 187.3 ± 40.5 | 173.8 ± 47.1 | 189.9 ± 27.8 | 200.1 ± 23.7 |

| | F1 | F2 | F3 | F4 | G | M7 | M7b | M8 | M10 | M21 |
|---|---|---|---|---|---|---|---|---|---|---|
| NUMBER | 51 | 14 | 7 | 9 | 11 | 17 | 50 | 35 | 9 | 1 |
| VALUE | 189.7 ± 38.7 | 198.8 ± 41.9 | 192.1 ± 30.3 | 192.2 ± 43.5 | 186.5 ± 47.6 | 200.5 ± 51.9 | 188.0 ± 43.7 | 196.6 ± 44.3 | 179 ± 18.8 | 196 |

| | M* | N9a | N* | R9 | R11 | R* | P |
|---|---|---|---|---|---|---|---|
| NUMBER | 14 | 17 | 1 | 13 | 3 | 1 | |
| VALUE | 183 ± 33.7 | 170 ± 37.8 | 268 | 206.7 ± 47.36 | 225 ± 40.7 | 244 | 0.07 |

The following tables provide correlations between phenotypes and haplotypes that have been grouped according to the legend below.

| | Macro D | Macro F | Macro R | Macro M |
|---|---|---|---|---|
| T2DM | 0.33 | 0.37 | 0.66 | 0.90 |
| Obesity | 0.60 | 0.52 | 0.46 | 0.62 |
| HTN | 0.14 | 0.62 | 0.69 | 0.08 |
| AHA MS | 0.91 | 0.83 | 0.29 | 0.41 |
| IDF MS | 0.59 | 0.90 | 0.23 | 0.56 |
| AC | 0.24 | 0.88 | 0.92 | 0.87 |
| PC | 0.67 | 0.60 | 0.36 | 0.17 |
| TG | 0.39 | 0.26 | 0.77 | 0.33 |
| T-chol | 0.71 | 0.72 | 0.54 | 0.90 |
| HDL-C | 0.19 | 0.47 | 0.52 | 0.81 |
| LDL-C | 0.53 | 0.50 | 0.29 | 0.78 |
| Waist | 0.66 | 0.78 | 0.79 | 0.60 |
| BMI | 0.32 | 0.70 | 0.86 | 0.40 |
| SBP | [symbol] | 0.94 | 0.47 | 0.05 |
| DBP | 0.17 | 0.84 | 0.76 | 0.53 |

“Macro M” refers to M7 + M7v + M8 + E + M10 + M21 + D + G + M*
“Macro F” refers to F1 + F2 + F3 + F4
“Macro R” refers to F1 + F2 + F3 + F4 + B + R9 + R11 + R*
“Macro D” refers to D + D* + D2 + D5 + D4b + D4a

The following table provides correlations between phenotypes and D2 vs. non-D2 haplotypes.

| | D2 | Non-D2 | P-value | OR (95% C.I.) |
|---|---|---|---|---|
| AGE | 43.18 ± 12.87 | 52.69 ± 14.02 | 0.03 | |
| SEX (M/F) | 5/6 | 215/268 | 0.98 | |
| TYPE 2 DIABETES | 3/8 | 224/252 | 0.23 | 0.42 (0.11-1.61) |
| Obesity (BMI > 30 kg/m2) | 3/8 | 108/354 | 0.73 | 1.23 (0.32-4.71) |
| Hypertension | 3/5 | 154/224 | 1.00 | 0.87 (0.21-3.71) |
| AHA MS | 5/3 | 197/145 | 1.00 | 1.23 (0.29-5.22) |
| IDF MS | 4/4 | 149/179 | 1.00 | 1.20 (0.30-4.90) |
| Fasting glucose | 114.18 ± 29.00 | 131.65 ± 50.67 | 0.46 | 0.83 (0.24-2.87) |
| (n = 11/474) | | | | |
| Postmeal glucose | 115.67 ± 26.70 | 164.59 ± 74.92 | 0.09 | 0.48 (0.12-1.92) |
| (n = 6/247) | | | | |
| Triglyceride (n = 11/470) | 166.27 ± 87.77 | 143.44 ± 106.31 | 0.21 | 1.63 (0.49-5.42) |
| Total cholesterol | 173.5 ± 24.30 | 191.83 ± 42.58 | 0.45 | Invalid value* |
| (n = 10/470) | | | | |
| HDL-C (n = 6/295) | 45.95 ± 7.52 | 48.37 ± 14.95 | 0.82 | 4.38 (0.48-39.64) |
| LDL-C (n = 2/132) | 81.8 ± 8.20 | 120.74 ± 34.00 | 0.09 | Invalid value* |
| Waist (n = 9/365) | 97.76 ± 20.85 | 94.92 ± 18.29 | 0.93 | 0.62 (0.16-2.36) |
| BMI (n = 11/462) | 28.71 ± 9.74 | 27.48 ± 7.49 | 0.98 | See obesity |
| SBP (n = 7/320) | 134.14 ± 30.66 | 128.82 ± 21.47 | 0.59 | 0.49 (0.12-2.07) |
| DBP (n = 7/320) | 77.14 ± 10.93 | 77.93 ± 11.32 | 0.84 | 0.39 (0.08-1.97) |

OR = 0

The following table provides correlations between phenotypes and D5 vs. non-D5 haplotypes.

| | D5 | Non-D5 | P-value | OR (95% C.I.) |
|---|---|---|---|---|
| AGE | 54.64 ± 12.96 | 52.31 ± 14.14 | 0.31 | |
| SEX (M/F) | 15/21 | 205/248 | 0.68 | |
| TYPE 2 DIABETES | 21/14 | 206/246 | 0.10 | 1.79 (0.89-3.61) |
| Obesity (BMI > 30 kg/m2) | 6/26 | 105/336 | 0.51 | 0.74 (0.30-1.84) |
| Hypertension | 13/18 | 144/211 | 0.88 | 1.06 (0.50-2.23) |
| AHA MS | 20/7 | 182/141 | 0.07 | 2.21 (0.91-5.38) |
| IDF MS | 14/8 | 139/175 | 0.08 | 2.20 (0.90-5.40) |
| Fasting glucose | 146.91 ± 62.55 | 130.03 ± 49.13 | 0.13 | 1.41 (0.64-3.08) |
| (n = 35/450) | | | | |
| Postmeal glucose | 204.33 ± 106.74 | 160.85 ± 71.51 | 0.09 | 2.94 (0.99-8.68) |
| (n = 15/238) | | | | |
| Triglyceride (n = 34/447) | 170.82 ± 119.21 | 141.92 ± 104.68 | 0.12 | 2.04 (1.01-4.11) |
| Total cholesterol | 215.11 ± 47.28 | 189.64 ± 41.45 | [symbol] | Invalid value* |
| (n = 34/446) | | | | |
| HDL-C (n = 26/275) | 49.13 ± 19.70 | 48.25 ± 14.34 | 0.95 | 1.51 (0.67-3.42) |
| LDL-C (n = 15/119) | 128.97 ± 38.32 | 119.05 ± 33.53 | 0.31 | 0.94 (0.30-2.93) |
| Waist (n = 24/350) | 96.49 ± 19.00 | 94.88 ± 18.30 | 0.68 | 1.99 (0.73-5.47) |
| BMI (n = 32/441) | 25.87 ± 6.68 | 27.63 ± 7.59 | 0.12 | See obesity |
| SBP (n = 26/301) | 137.54 ± 27.33 | 128.19 ± 20.98 | 0.056 (0.047) | 1.35 (0.60-3.06) |
| DBP (n = 26/301) | 79.07 ± 12.97 | 77.81 ± 11.15 | 0.42 | 1.43 (0.64-3.19) |

*OR = 0

The following table provides correlations between phenotypes and D4a vs. non-D4a haplotypes.

| | D4a | Non-D4a | P-value | OR (95% C.I.) |
|---|---|---|---|---|
| AGE | 49.64 ± 16.47 | 52.61 ± 13.93 | 0.33 | |
| SEX (M/F) | 14/9 | 206/260 | 0.12 | |
| TYPE 2 DIABETES | 12/10 | 215/250 | 0.44 | 1.40 (0.59-3.30) |
| Obesity (BMI > 30 kg/m2) | 8/15 | 103/347 | 0.19 | 1.80 (0.74-4.36) |
| Hypertension | 10/6 | 147/223 | 0.07 | 2.53 (0.910-7.11) |
| AHA MS | 8/8 | 194/140 | 0.52 | 0.72 (0.26-1.97) |
| IDF MS | 4/12 | 149/171 | 0.12 | 0.38 (0.12-1.21) |
| Fasting glucose | 135.45 ± 44.95 | 131.05 ± 50.60 | 0.47 | 1.02 (0.41-2.56) |
| (n = 22/463) | | | | |
| Postmeal glucose | 163.71 ± 73.49 | 163.4 ± 74.7 | 0.81 | 1.03 (0.41-2.60) |
| (n = 14/239) | | | | |
| Triglyceride (n = 22/459) | 112.82 ± 50.08 | 145.45 ± 107.65 | 0.15 | 0.90 (0.36-2.25) |
| Total cholesterol | 178.52 ± 30.60 | 192.04 ± 42.74 | 0.18 | Invalid value* |
| (n = 21/459) | | | | |
| HDL-C (n = 17/284) | 51.32 ± 11.75 | 48.15 ± 15.00 | 0.41 | 0.57 (0.20-1.57) |
| LDL-C (n = 5/129) | 94.16 ± 11.11 | 121.17 ± 34.39 | 0.10 | Invalid value* |
| Waist (n = 20/354) | 98.21 ± 22.39 | 94.81 ± 18.09 | 0.90 | 0.39 (0.16-0.97) |
| BMI (n = 23/450) | 28.62 ± 8.81 | 27.45 ± 7.47 | 1.00 | See obesity |
| SBP (n = 15/312) | 136.33 ± 27.07 | 128.58 ± 21.34 | 0.23 | 1.86 (0.63-5.48) |
| DBP (n = 15/312) | 76.2 ± 11.53 | 77.99 ± 11.30 | 0.49 | 2.06 (0.73-5.79) |

*OR = 0

The following table provides correlations between phenotypes and D4b vs. non-D4b haplotypes.

| | D4b | Non-D4b | P-value | OR (95% C.I.) |
|---|---|---|---|---|
| AGE | 52.03 ± 15.31 | 52.49 ± 14.04 | 0.92 | |
| SEX (M/F) | 8/4 | 212/265 | 0.11 | |
| TYPE 2 DIABETES | 5/7 | 222/253 | 0.78 | 0.81 (0.26-2.60) |
| Obesity (BMI > 30 kg/m2) | 2/10 | 109/352 | 0.74 | 0.65 (0.14-2.99) |
| Hypertension | 5/5 | 152/224 | 0.54 | 1.47 (0.42-5.28) |
| AHA MS | 3/6 | 199/142 | 0.17 | 0.36 (0.09-1.45) |
| IDF MS | 3/8 | 150/175 | 0.35 | 0.44 (0.11-1.68) |
| Fasting glucose | 135 ± 58.14 | 131.16 ± 50.18 | 0.90 | 0.95 (0.28-3.20) |
| (n = 12/473) | | | | |
| Postmeal glucose | 164.33 ± 90.44 | 163.40 ± 74.28 | 0.89 | 1.13 (0.29-4.43) |
| (n = 6/247) | | | | |
| Triglyceride (n = 12/469) | 83.03 ± 25.64 | 145.52 ± 106.72 | [symbol] | Invalid value* |
| | | | [symbol] | |
| Total cholesterol | 187.25 ± 40.43 | 191.56 ± 42.43 | 0.84 | Invalid value* |
| (n = 12/468) | | | | |
| HDL-C (n = 7/294) | 55.6 ± 20.47 | 48.15 ± 14.68 | 0.23 | 0.42 (0.08-2.20) |
| LDL-C (n = 3/131) | 100.37 ± 28.89 | 120.62 ± 34.15 | 0.30 | Invalid value* |
| Waist (n = 11/363) | 92.78 ± 19.67 | 95.05 ± 18.31 | 0.52 | 0.60 (0.18-1.99) |
| BMI (n = 12/461) | 27.06 ± 10.99 | 27.52 ± 7.44 | 0.39 | See obesity |
| SBP (n = 9/318) | 137.11 ± 23.46 | 128.70 ± 21.59 | 0.25 | 1.25 (0.35-4.49) |
| DBP (n = 9/318) | 78.56 ± 14.21 | 77.89 ± 11.23 | 0.71 | 1.21 (0.34-8.24) |

*OR = 0

The following table provides correlations between phenotypes and D4b(T) vs. non-D4b(T) haplotypes.

| | D4b (T) | Non-D4b (T) | P-value | OR (95% C.I.) |
|---|---|---|---|---|
| AGE | 57.1 ± 10.85 | 52.38 ± 14.11 | 0.29 | |
| SEX (M/F) | 8/2 | 212/267 | 0.048 | |
| TYPE 2 DIABETES | 5/5 | 222/255 | 1.00 | 1.15 (0.33-4.02) |
| Obesity (BMI > 30 kg/m2) | 0/10 | 111/352 | 0.13 | Invalid value** |
| Hypertension | 5/5 | 152/224 | 0.54 | 1.47 (0.42-5.18) |
| AHA MS | 2/5 | 200/143 | 0.14 | 0.30 (0.06-1.50) |
| IDF MS | 2/7 | 151/176 | 0.19 | 0.33 (0.07-1.63) |
| Fasting glucose | 142.5 ± 61.00 | 131.02 ± 50.13 | 0.73 | 1.12 (0.28-4.26) |
| (n = 10/475) | | | | |
| Postmeal glucose | 178 ± 106.22 | 163.19 ± 74.15 | 0.82 | 1.45 (0.29-7.30) |
| (n = 4/249) | | | | |
| Triglyceride (n = 10/471) | 82.9 ± 28.34 | 145.26 ± 106.56 | [symbol] | Invalid value* |
| | | | [symbol] | |
| Total cholesterol | 198.8 ± 33.27 | 191.29 ± 42.53 | 0.58 | Invalid value* |
| (n = 10/470) | | | | |
| HDL-C (n = 5/296) | 55.64 ± 23.46 | 48.20 ± 14.68 | 0.35 | 0.26 (0.03-2.38) |
| LDL-C (n = 3/131) | 100.37 ± 28.88 | 120.62 ± 34.15 | 0.30 | Invalid value* |
| Waist (n = 9/365) | 85.82 ± 7.72 | 95.21 ± 18.46 | 0.11 | 0.39 (0.10-1.49) |
| BMI (n = 10/463) | 23.38 ± 3.18 | 27.59 ± 7.58 | 0.07 | See obesity |
| SBP (n = 9/318) | 137.11 ± 23.46 | 128.70 ± 21.59 | 0.25 | 1.25 (0.35-4.49) |
| DBP (n = 9/318) | 78.56 ± 14.21 | 77.89 ± 11.23 | 0.71 | 1.21 (0.34-4.24) |

OR = 0

The following table provides correlations between phenotypes and D⁺ vs. non-D⁺ haplotypes.

| | D* | Non-D* | P-value | OR (95% C.I.) |
|---|---|---|---|---|
| AGE | 57.67 ± 12.67 | 52.35 ± 14.08 | 0.19 | |
| SEX (M/F) | 7/5 | 213/264 | 0.39 | |
| TYPE 2 DIABETES | 8/4 | 219/256 | 0.24 | 2.34 (0.70-7.87) |
| Obesity (BMI > 30 kg/m2) | 4/8 | 107/354 | 0.49 | 1.66 (0.49-5.60) |
| Hypertension | 5/4 | 152/225 | 0.50 | 1.85 (0.49-7.00) |
| AHA MS | 6/4 | 196/144 | 1.00 | 1.10 (0.31-3.97) |
| IDF MS | 4/5 | 149/178 | 1.00 | 0.96 (0.25-3.62) |
| Fasting glucose | 141.92 ± 50.86 | 130.99 ± 50.33 | 0.23 | 2.42 (0.52-11.19) |
| (n = 12/473) | | | | |
| Postmeal glucose | 196.17 ± 52.88 | 162.63 ± 74.84 | 0.10 | 3.93 (0.49-31.78) |
| (n = 6/247) | | | | |
| Triglyceride (n = 12/469) | 136.08 ± 78.20 | 144.16 ± 106.57 | 0.91 | 0.97 (0.29-3.26) |
| Total cholesterol | 189.92 ± 27.82 | 191.49 ± 42.67 | 0.75 | Invalid value* |
| (n = 12/468) | | | | |
| HDL-C (n = 7/294) | 45.73 ± 8.27 | 48.39 ± 14.96 | 0.74 | 1.44 (0.32-6.55) |
| LDL-C (n = 4/130) | 126.2 ± 25.43 | 119.98 ± 34.37 | 0.52 | 1.92 (0.26-14.18) |
| Waist (n = 10/364) | 98.22 ± 19.27 | 94.89 ± 18.32 | 0.50 | 1.18 (0.30-4.65) |
| BMI (n = 12/461) | 28.73 ± 8.27 | 27.46 ± 7.53 | 0.76 | See obesity |
| SBP (n = 9/318) | 140.11 ± 22.21 | 128.61 ± 21.59 | 0.15 | 1.67 (0.41-6.79) |
| DBP (n = 9/318) | 85.44 ± 12.11 | 77.69 ± 11.22 | 0.053 | 1.51 (0.40-5.73) |

*OR = 0

The following table provides correlations between phenotypes and women with D5 haplotypes vs. women with non-D5 haplotypes.

| | Women with D5 | Women without D5 | P-value | OR (95% C.I.) |
|---|---|---|---|---|
| AGE | 54.64 ± 12.96 | 52.31 ± 14.14 | 0.62 | |
| TYPE 2 DIABETES | 13/7 | 117/131 | 0.13 | 2.07 (0.80-5.39) |
| Obesity (BMI > 30 kg/m2) | 6/13 | 72/167 | 0.89 | 1.01 (0.39-2.93) |
| Hypertension | 6/10 | 74/106 | 0.78 | 0.86 (0.30-2.47) |
| AHA MS | 12/2 | 113/67 | 0.09 | 3.66 (0.80-16.86) |
| IDF MS | 11/3 | 96/77 | 0.16 | 2.94 (0.79-10.9) |
| Fasting glucose | 146.91 ± 62.55 | 130.04 ± 49.13 | 0.08 | 1.84 (0.59-5.67) |
| (n = 35/450) | | | | |
| Postmeal glucose | 204.33 ± 106.74 | 160.85 ± 71.51 | 0.10 | 6.67 (0.86-51.62) |
| (n = 15/238) | | | | |
| Triglyceride (n = 34/447) | 170.82 ± 119.21 | 141.92 ± 104.68 | 0.11 | 1.95 (0.76-4.99) |
| Total cholesterol | 215.12 ± 47.28 | 189.64 ± 41.45 | 0.015 (0.09) | Invalid value* |
| (n = 34/446) | | | | |
| HDL-C (n = 26/275) | 49.14 ± 19.70 | 48.25 ± 14.34 | 0.69 | 1.39 (0.46-4.19) |
| LDL-C (n = 15/119) | 128.97 ± 38.32 | 119.05 ± 33.53 | 0.33 | 1.11 (0.23-5.37) |
| Waist (n = 24/350) | 96.50 ± 19.00 | 94.88 ± 18.30 | 0.27 | 3.67 (0.47-28.57) |
| BMI (n = 32/441) | 25.87 ± 6.68 | 27.63 ± 7.59 | 0.95 | See obesity |
| SBP (n = 26/301) | 137.54 ± 27.34 | 128.19 ± 20.98 | 0.04 (0.011*) | 1.72 (0.51-5.79) |
| DBP (n = 26/301) | 79.08 ± 12.97 | 77.81 ± 11.16 | 0.20 | 1.42 (0.36-4.42) |

*OR = 0

The following table provides correlations between phenotypes and men with D5 haplotypes vs. men with non-D5 haplotypes.

| | Men with D5 | Men without D5 | P-value | OR (95% C.I.) |
|---|---|---|---|---|
| AGE (N = 36/451) | 54.64 ± 12.96 | 52.31 ± 14.14 | 0.03 | |
| TYPE 2 DIABETES | 8/7 | 88/115 | 0.45 | 1.49 (0.52-4.28) |
| Obesity (BMI > 30 kg/m2) | 0/13 | 33/169 | 0.23 | — |
| Hypertension | 7/8 | 70/105 | 0.79 | 1.31 (0.46-3.78) |
| AHA MS | 8/5 | 69/72 | 0.56 | 1.67 (0.52-5.35) |
| IDF MS | 3/5 | 43/98 | 0.70 | 1.37 (0.31-5.98) |
| Fasting glucose | 146.91 ± 62.55 | 130.04 ± 49.13 | 0.87 | 1.05 (0.35-3.20) |
| (n = 35/450) | | | | |
| Postmeal glucose | 204.33 ± 106.74 | 160.85 ± 71.51 | 0.50 | 1.61 (0.41-6.30) |
| (n = 15/238) | | | | |
| Triglyceride (n = 34/447) | 170.82 ± 119.21 | 141.92 ± 104.68 | 0.65 | 2.17 (0.76-6.23) |
| Total cholesterol | 215.11 ± 47.28 | 189.64 ± 41.45 | 0.012 (0.031) | Invalid value |
| (n = 34/446) | | | | |
| HDL-C (n = 26/275) | 49.13 ± 19.69 | 48.24 ± 14.34 | 0.75 | 1.69 (0.51-5.64) |
| LDL-C (n = 15/119) | 128.97 ± 38.32 | 119.05 ± 33.53 | 0.66 | 0.88 (0.16-4.89) |
| Waist (n = 24/350) | 96.49 ± 19.00 | 94.88 ± 18.30 | 0.21 | 0.88 (0.19-4.04) |
| BMI (n = 32/441) | 25.87 ± 6.67 | 27.63 ± 7.59 | 0.0048 (0.030*) | See obesity |
| SBP (n = 26/301) | 137.54 ± 27.34 | 128.19 ± 20.98 | 0.46 | 1.09 (0.35-3.37) |
| DBP (n = 26/301) | 79.07 ± 12.97 | 77.81 ± 11.16 | 0.92 | 1.44 (0.47-4.47) |

### EXAMPLES

The following example is offered to illustrate a method of making a genetic diagnosis of an mtDNA-based disease. Though tests for the genetic diagnosis of Leber's Hereditary Optic Neuropathy are described in the following example, the methods are also applicable for the development of tests for the genetic diagnosis of Metabolic Syndrome and/or phenotypes of Metabolic Syndrome. The example is meant to further illustrate but not to limit the invention.

### A Complete Strategy for Excluding Mitochondrial DNA in the Clinical Diagnosis of Common Mitochondrial Diseases

Mitochondria are the primary energy source of cells and contain their own unique extra-chromosomal DNA. Polymorphisms arising in the mitochondrial genome (mtDNA) are part of normal genetic variability in humans. The compactness, abundance and elevated mutation rate of mtDNA compared to nuclear DNA (nDNA) make it useful for tracking human migration, forensic identification and diagnosing disease. The diseases arising from mtDNA range in severity from tiredness and muscle weakness to sudden onset blindness, seizure or stroke-like episodes and are a frequent cause of infant death. Unfortunately, clinical genetic diagnosis of mitochondrial disease by matching a phenotype to its underlying mutation is not always straightforward. A single mutation can present with a range of phenotypes (such as MELAS) whereas different mutations can sometimes result in the same phenotype (such as Leber's) thus making genetic diagnosis difficult. Here we present a complete strategy and system for the genetic diagnosis of mtDNA based disease. This is achieved through the simultaneous screening of multiple mutations based on the most common disease phenotypes, followed by a series of downstream analysis steps. Using this strategy should result in a definitive determination that an abnormality in mtDNA exists or alternatively that it may be excluded from consideration as the primary cause of illness.

Mitochondrial pathologies are complex diseases the diagnoses of which currently require both biochemical and genetic characterization of the patient. Confirming a specific diagnosis of mitochondrial disease is complicated by the fact that a single mutation is able to present a host of different symptoms that vary with the mutational load and tissue distribution. The tissue-specific manifestations of these diseases result from the varying energetic needs of different tissues (Wallace, 2005). Because mitochondrial DNA (mtDNA) is present in multiple copies, up to 1000's per cell, diagnosis often requires the use of a muscle biopsy, enzymatic or biochemical assay to determine the level and severity of the defect found in the patient. This is often followed by a partial or complete sequencing of the mitochondrial genome or by testing individual SNP loci using methods that can quantitatively measure mutation levels. Existing methods for the genetic diagnosis of mitochondrial disease include direct sequencing, specific locus amplification followed by RFLP analysis, quantification with dHPLC heteroduplex formation, fluorescent based primer extension and qPCR (Cassandrini, et al., 2006; Genasetti, et al., 2007; Procaccio, et al., 2006). Traditional test methods like RFLP can be combined with newer techniques such as dHPLC to quantify heteroplasmic levels and will continue to be useful in mitochondrial disease diagnosis, but have limitations that need to be overcome. RFLP requires the detection of just one polymorphism at a time when the number of pathological mutations being discovered on mtDNA has increased to over 100 and its heteroplasmy detection limit is around 8% depending on the site. On the other hand, microarray analysis can test all sites simultaneously but is costly and does not yet have the sensitivity needed for accurate heteroplasmic detection. RFLP analysis has the added drawback of requiring PCR amplification that could skew results in difficult to amplify regions that have a high GC content or strong folding tendencies. Unequal PCR amplification efficiency in cases with heteroplasmic mtDNA can hamper accurate diagnosis and affect treatment options or proper diagnosis. This is particularly important for mtDNA since much of its sequence contains regions with high tendency for secondary structure due to the presence of an entire set of self-folding tRNA and two rRNA sequences. Currently there are several dozen confirmed and several hundred provisional disease polymorphisms found in the mitochondrial genome alone and there remains a need for further refinement of diagnostic techniques to improve efficiency and simplify analysis of mitochondrial disease diagnosis.

To deal with the increasing complexity of mitochondrial disease genetics, we have developed a clinical diagnostic tool around an expandable primer extension based technique, the SNaPshot assay from Applied Biosystems. Through multiplexing primers targeted to disease causing SNPs this tool can be used to simultaneously interrogate all of the most common pathogenic mtDNA polymorphisms (see http://www.mitomap.org for a complete list). It can also be used in quantitative detection of heteroplasmic mutations by plotting it to a standard curve (Cassandrini, et al., 2006). In addition to testing the 13 most common polymorphisms we demonstrate an additional phenotype driven model of disease diagnosis through the development of a test containing the 10 most common Lebers Hereditary Optic Neuropathy (LHON) mutations including a test for the most common haplotypes of those carrying this disease, the J and T haplogroups (Brown, et al., 1997).

Together these tests form the initial analysis of a larger strategy designed to simplify the diagnosis of mitochondrial genetic disease. In this strategy, patient DNA samples sent from a referring clinician are PCR amplified followed by SNP detection using a fluorescent multiplex primer extension assay designed to detect the most common pathologies or through a focused panel associated with the patient's prevalent phenotypes. If a mutation is detected, the percent heteroplasmy is determined for that nucleotide using, primer extension, a FAM labeling assay or qPCR. If no mutation is discovered then alternate means for screening the entire mitochondrial genome are pursued to look for less common mutations, insertions, or deletions.

### Materials and Methods

SNP Site Selection

The selected polymorphisms along with associated pathologies were tested. The numbering of test sites followed the revised Cambridge Reference Sequence of mtDNA (Genbank Accession J01415.2 GI:113200490). Disease sites were selected based on clinical phenotypes (non-LHON) associated with mtDNA polypeptide gene mutations as published at http://www.mitomap.org/rimtab2.html and from the Leber's Hereditary Optic Neuropathy table published at http://www.mitomap.org/rimtab1.html for the LHON phenotype assay.

Primers

Extension primers were designed for amplification and extension based on the revised Cambridge Reference Sequence of mtDNA. We developed a custom primer design program to identify primers with melting temperatures between 53° C. and 57° C. Primer lengths for the final version of the assay fall between 15 and 70 bp in length and were selected to differ by 3-5 bp each to prevent spatial overlap for sites incorporating the same nucleotide. In certain cases where overlap still occurred, poly(T) tails were added to primers to adjust their elution point for use in multiplexing and to create non-overlapping bins for use in automating base calls with the Genemapper Software v4.0 (Applied Biosystems Inc., Foster City, Calif.). Primers were ordered from Integrated DNA Technologies (Iowa City, Iowa). Primers over 38 bp were ordered PAGE purified. All primers were stored in 100 uM stocks in 10 mM Tris 0.5 mM EDTA pH 8.0 at −20 C for long-term storage.

Sample Preparation and Process Strategy

DNA was obtained from a variety of sources including buccal swab, muscle, lymphoblast and 143B fibroblast cell lines. Genomic DNA was isolated from either blood or lymphoblast cells using the puregene DNA isolation kit (Gentra Systems, Qiagen). For some experiments, mtDNA was isolated using the mtDNA isolation kit according to the manufacturer's instructions (Biovision Research Products Inc. Mountain View, Calif.). For assay validation of the disease and haplotyping panels, samples were randomized in a blind control study. Following initial diagnosis, primer extension information was compared to the original sequence data and other verification data to confirm the consistency of the primer extension data and to show that the overall strategy could successfully type samples of unknown origin.

mtDNA Amplification (Whole Genome—7 Fragment Coding Plus 1 D-Loop Fragment)

Whole mitochondrial genome amplification was accomplished through PCR in 8 overlapping fragments ranging in size from 1500 bp to 2500 bp. Following amplification fragments are ExoSAP-IT treated and then spiked in to the extension reaction as a mix. This complete coding region amplification strategy was chosen to allow for easy expansion and addition of specific test sites from anywhere in the mitochondrial genome without the need to redesign amplicons. All primer sets have previously been tested and found negative for pseudogene amplification using a rho zero cell line lacking mitochondrial DNA. To amplify mtDNA fragments we used between 25-50 ng of genomic DNA as starting material along with 0.4 umol primer 1, 0.4 umol primer 2, 50 uM dNTPs and 0.25 ul (1 U) high concentration Roche Taq polymerase. Amplification conditions were 94° C. 5 min (94° C. 45 seconds/56° C. 30 seconds/72° C. 3 minutes) 35 cycles/72° C. 5 minutes/4° C.∞

Multiplex PCR Conditions

To increase assay throughput and reduce assay cost, the 7 coding region amplification products were combined into two non-overlapping multiplex reactions. Odd numbered fragments were added to multiplex 1 and even numbered fragment into multiplex 2. Other optimizations included increasing the polymerase to 3 U/rxn and the magnesium concentration to 2.5 mM final.

Primer Extension

Primer extension reactions were performed essentially as in (Vallone, et al., 2004) Briefly, 0.2 uM each of extension primers were added to ExoSAP-IT purified PCR products, 5 ul of 2× SNaPshot mastermix including polymerase and labeled ddNTPs and 1× reaction buffer in a total volume of 10 ul. The mixture was incubated at 94° C. for a 1 minute denaturation followed by 25-40 cycles at 94° C. for 10 seconds and 53° C. for 5 and 60° C. for 10 minutes and a final denaturation at 94° C. for 30 seconds. Before running on the ABI 3130xl instruments GeneLiz120 size standard was added to samples diluted 1:10 in Hi-Di formamide and denatured for 5 minutes at 95 C and then immediately chilled on ice.

PCR Product Purification and Cleanup

Post PCR amplification products were treated with ExoSAP-IT (USB Corporation) for 15 minutes at 37° C. followed by inactivation for 20 minutes at 80° C. to remove any remaining dNTPs that might interfere with subsequent primer extension steps. Post extension reactions were cleaned with Shrimp Alkaline Phosphate for 15 minutes at 37° C. followed by a denaturation at 70° C. for 10 minutes

Primer Generation Program

Software was developed to identify candidate primers that could be used to analyze a nucleotide position of interest. Ranges for primer melting temperature and salt concentration, a list of positions, and the reaction concentrations for salt and oligonucleotides are submitted as parameters to the program. Candidate primers having a predicted melting temperature within the range submitted are then generated for each position in the list using the equation. Primer candidates are based on the revised Cambridge Reference Sequence. The program is accessible through a website interface: http://mammag.web.uci.edu/twiki/bin/view/Mitomaster/AnalysisPrimerSelection.

DNA Collection, Purification and Quantification

Sensitivity experiments were performed on serial dilutions of DNA quantified by spectrophotometry at 260 nm and confirmed through gel electrophoresis. PCR product quantification was performed using a Bio-Rad gel GelDocXR imager (Bio-Rad Inc., Hercules, Calif.). The PCR amplification product was run on an agarose gel alongside a standard DNA ladder of known quantity (typical the O-gene Ruler plus—Fermentas). Image analysis was performed with the Quantity One™ software from Bio-Rad and normalized over several iterations of gels. A consistency check for this method was performed from those same amplification products using pico green dye measured against a DNA standard curve and read using a NOVOstar fluorescent plate reader

Analysis of Extension Reactions with Genemapper

Bins for the automated calling of control and pathogenic polymorphisms using Genemapper 4.0 were set up. Along with the multiplex amplifications and extension reactions, this greatly facilitated the ability to run samples in high-throughput mode and allows for fast typing of individual haplotypes or mutation detection.

### Results

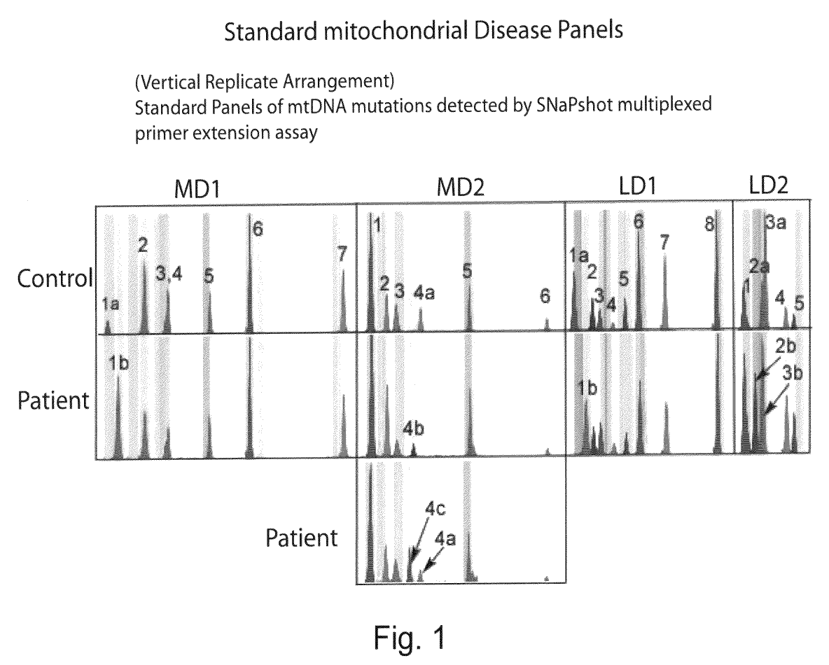

Standard peak distributions for a healthy individual are profiled in FIG. 1. The generalized assay was designed to detect the 13 most common pathogenic mitochondrial DNA mutations from selections based on information described at http://www.mitomap.org. The LHON assay detects the 10 most prevalent Leber's mutations along with coding region polymorphisms identifying haplogroups J, T and macrogroup JT, the haplogroups most commonly diagnosed with Leber's. Similarly the LHON sites were chosen from the 10 most commonly identified LHON sites and 3 haplotype positions (Brown, et al., 1992a; Brown, et al., 1992b; Brown, et al., 1992c; Wallace, et al., 1992). The SNPs for both assays are primarily biallelic with the exception of four positions: the 7445 deafness site, 8993 NARP, 3243 MELAS and 14482 LHON positions where an additional allele is known to have occurred in some patients (Goto, et al., 1991; Holt, et al., 1990; Luberichs, et al., 2002; Reid, et al., 1994; Tanaka, et al., 1991; Valentino, et al., 2002). Extension primers in use range in size from 18 to 70 bases in length. Once bins have been established and analysis parameters have been defined, the genemapper software automatically determines the presence of a control or mutant peak for each polymorphism. This is achieved through bin settings established during assay optimization and can be seen here as faint pastel colored bands.

Because some mtDNA mutations can be present at low percentages in some patients and in a tissue dependent manner, we wanted to determine the limits of detection for heteroplasmy at a pathogenic site. This also helped determine the possibility of heteroplasmic quantification of the SNP sites. We chose the 3243 site to perform quantification due to the availability of vector cloned control and patient DNA and the availability of clonal populations of a 143B cell line containing previously characterized 3243 heteroplasmy levels (Chomyn, et al., 1992; Procaccio, et al., 2006). In this way the validity of our results have been statistically compared to several independent quantification methods (FAM labeling, DHPLC, Real-Time (3460 mutation genesetti paper), pyro-sequencing). (This is also consistent with Cassandrini et al 2006). Following the generation of a standard curve from the 3243 site. We assayed several 143B cybrids containing variable heteroplasmy at the tRNAleu 3243 mutation. The results were compared against a recently developed and sensitive DHPLC based method for measuring heteroplasmic mutations known as the PARFAH method (Procaccio, et al., 2006).

When generating standard curves for working primers from known Wild-type and Mutant fragments for the 3243 primer, we found the extension reaction itself to be highly robust and that assay sensitivity could be improved for low heteroplasmy levels by increasing the sequencer's injection voltage. Using known quantities of wild type and mutant DNA, we were able to consistently detect heteroplasmy levels down to 0.01% of total DNA. Signal saturation was dealt with through diluting the extension reaction after amplification and before sequencing. The only potential drawback from this optimization is that of slightly increased background signal or interference from background peaks in the case of low heteroplasmy levels. Other optimizations for assay sensitivity include increasing the extension cycle number, altering primer sequencing to improve binding efficiency and specificity, and increasing or decreasing primer concentrations.

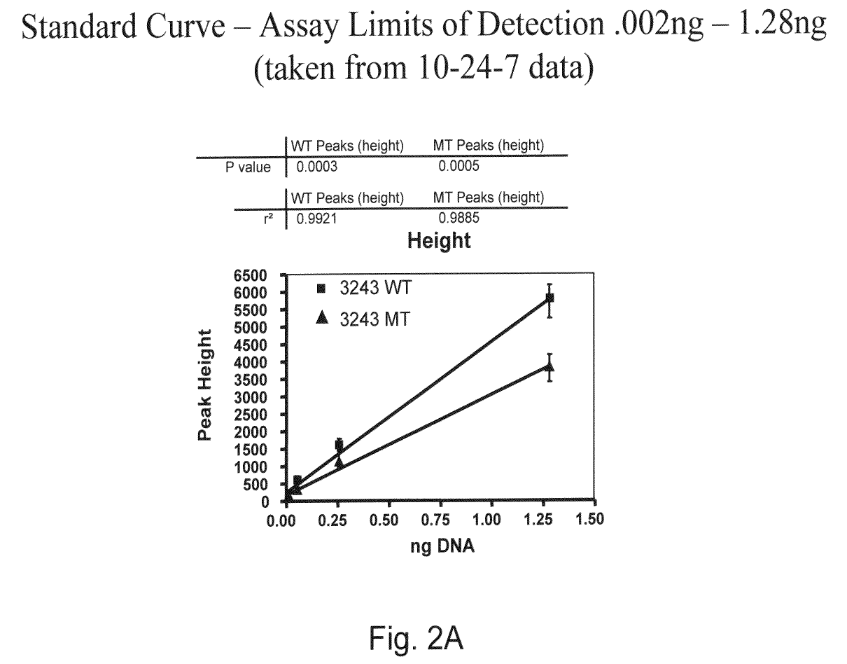

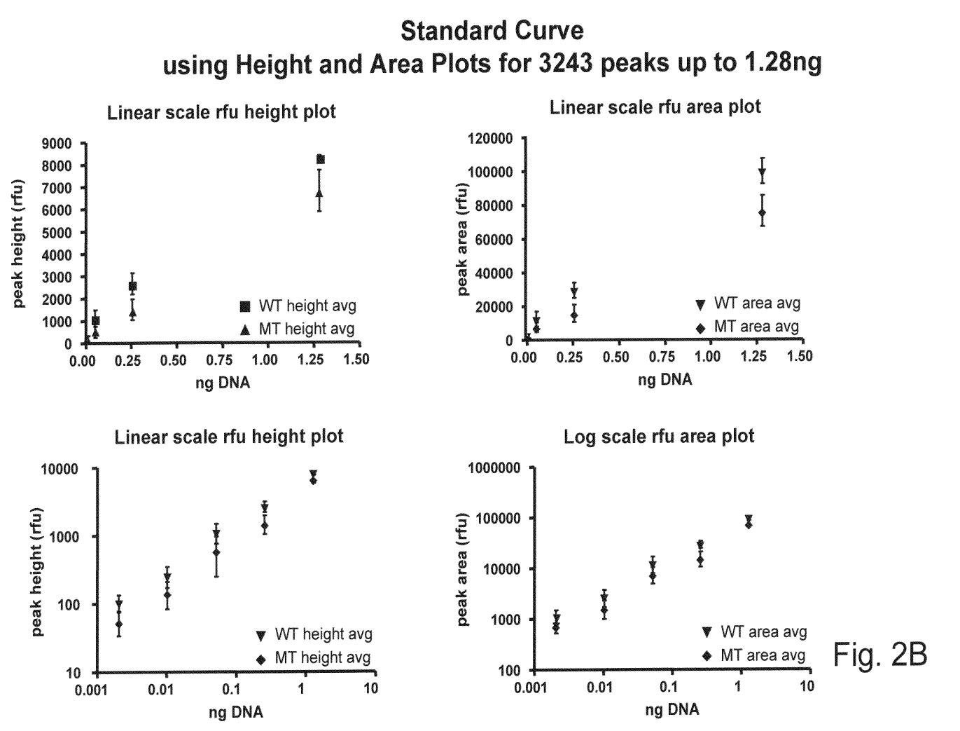

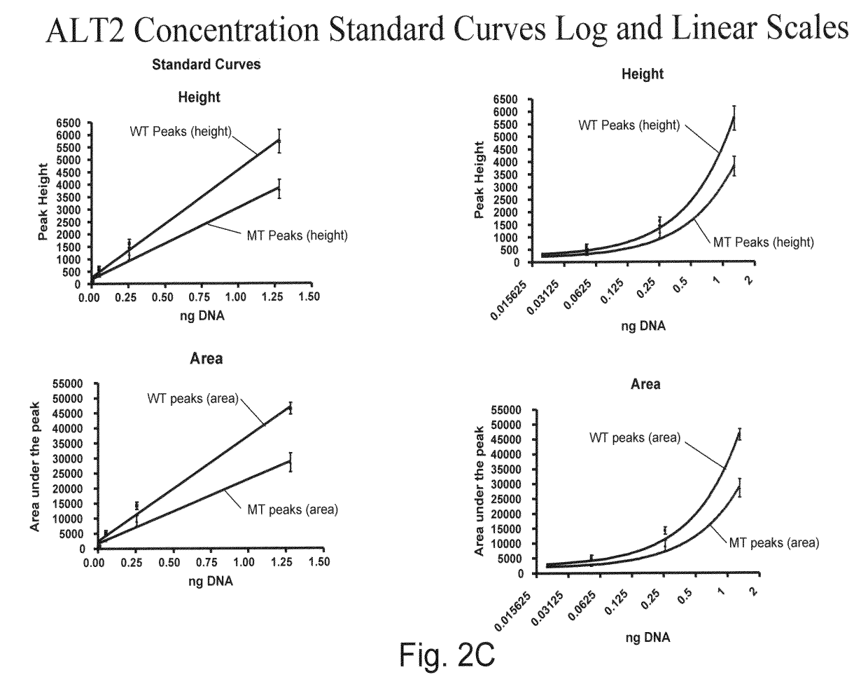

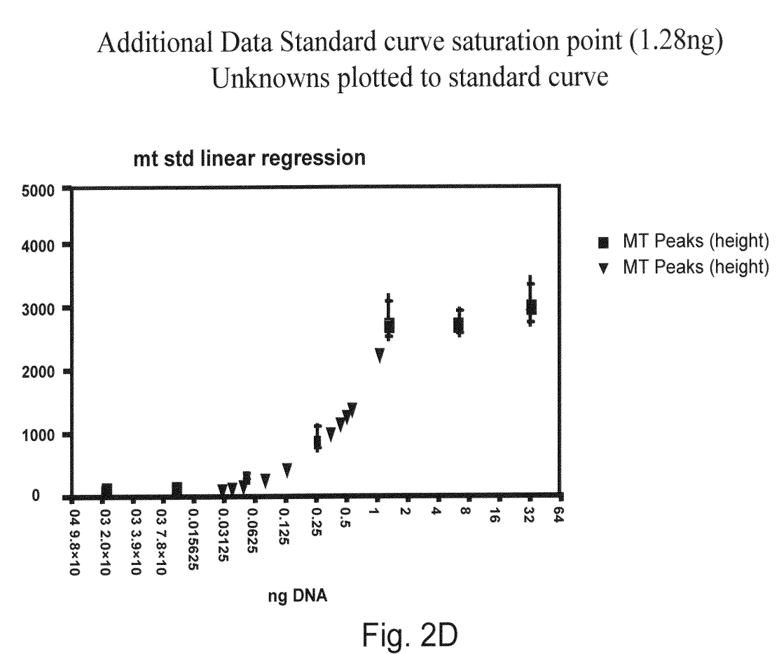

FIG. 2 depicts the limits of detection (32 ng-0.002 ng singleplex) and assay sensitivity for 3243 mutations. When performing standard curves we show that values in our curve above 1.28 ng (3.26e+9 copies) were saturated and the extension results were most consistent using starting DNA concentrations in the range of 0.01 to 1 ng for the 364 base pair product (2.55e+7 to 2.5e+9 copies). By optimizing our starting material and determining copy number we were able to achieve a goodness of fit r² values of 0.96 for the WT regression curve and 0.94 for the MT curve with p values of <0.0001 suggesting a significant linear correlation between starting DNA quantities and the resultant relative fluorescence. These values agree well with the Cassandrini paper which published similar R² values of 0.9399 for the 3243 position.

By extrapolating the copy number, the SNaPshot assay has an effective range of two orders of magnitude when following the manufacturer provided protocols for SNaPshot and the default fragment analysis method of the sequencer. This range will shift slightly depending on the efficiency of primer binding, extension cycle number, sequencing conditions etc, but this data provides a standard starting range for most assays.

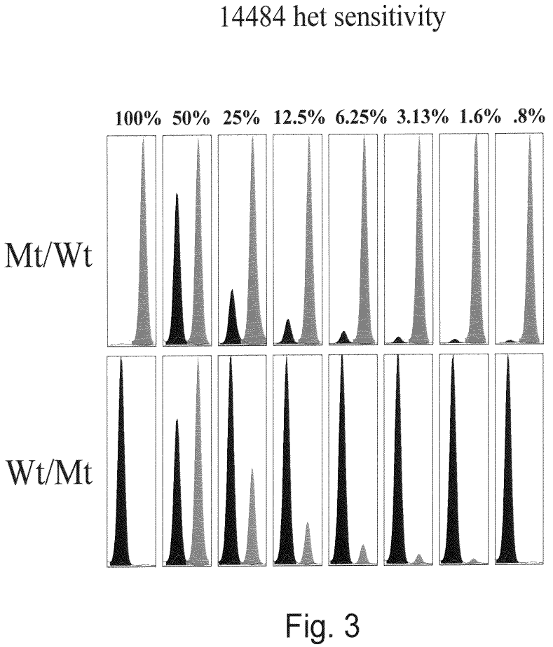

3243 disease quantification (with 143B DNA) is shown in FIG. 3. Using the standard curves generated in figure two we tested the validity of quantitating heteroplasmic mutations using previously characterized 143B cells harboring the 3243 mutation at varying levels. These samples have been previously characterized using both dHPLC and pyrosequencing methods.

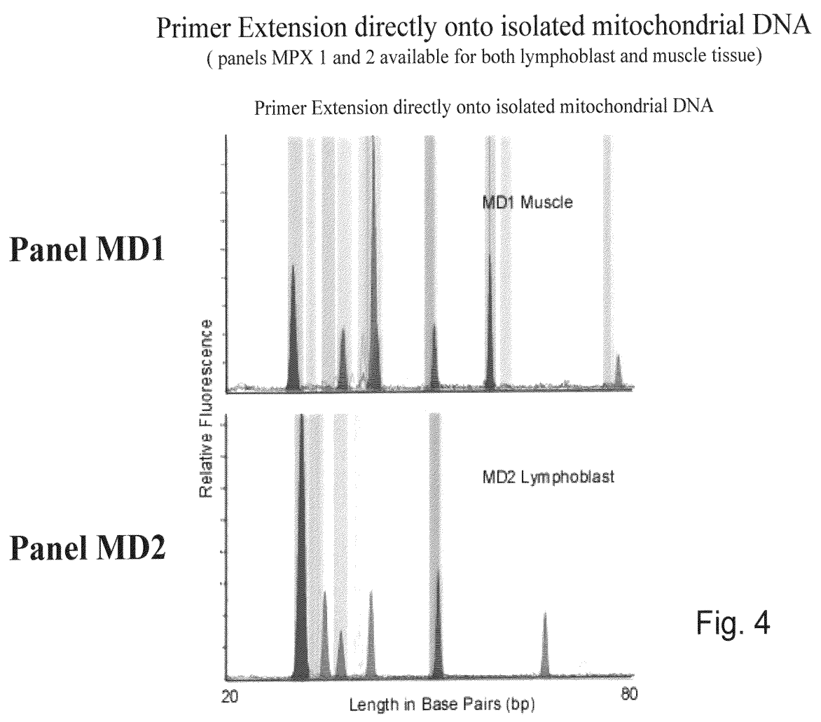

A unique aspect of mtDNA among most higher eukaryotes is the fact that it encodes the whole cohort of 22 tRNAs and two rRNAs in a compact genetic space of 16 kb due to its archaic (vestigial) bacterial genetic code. This creates the potential for a whole host of secondary structure in mtDNA. These tRNA and rRNA sequences, due to their hairpin loop forming sequences are able to fold spontaneously into their native conformation. DNA tends to have a higher stability than RNA given the same sequence, but still demonstrates a folding tendency, a result of a more favorable free energy of its folded state. This folding tendency has implications for both mitochondrial disease and its diagnosis. For disease, the risk is that the pathology can arise due to alternative folding affected by sequence alterations occurring in or proximal to the tRNAs as occurs in mutations such as the MELAS mutations. Similarly, diagnosis can be adversely affected through sequence changes that alter polymerase processivity or fidelity, skewing estimates of wildtype to mutant mtDNA ratios. Indeed, designing a primer for the 3243 and 8993 sites proved challenging and required numerous revisions due to nucleotide misincorporation in healthy samples. To overcome this issue we utilized the high sensitivity of fluorescent nucleotides and high copy number of mtDNA present in many tissues to eliminate the amplification step from our assay. By direct isolation of plasmid mtDNA from sample cells and tissues we have were able to extend primer probes directly on mtDNA, hopefully reducing variability in our measurements and providing greater accuracy when estimating heteroplasmy. FIG. 4 shows the results of primer extension directly off of purified isolated mtDNA from both a muscle biopsy (FIG. 4a) and a patient derived lymphoblast line (FIG. 4b). The gel in FIG. 4c shows the purified mtDNA band migrating at the expected 16 kb range for both the lymphoblast and muscle samples and mtDNA specific primers easily amplified their target fragments using this extract as a template. While the result of this procedure is very clean with low background there is likewise a very low signal, which we attempted to overcome in several ways. The most obvious solution is to use more mtDNA, but this may not be possible depending on tissue availability. Other ways to increase the signal level is by spiking additional primer into the reaction for lower peaks, adding additional extension cycles and manipulating the sequencer through a longer injection time and higher injection voltage. In this example we used 50 extension cycles instead of the normal 25 and an injection voltage of 2.2 v instead of the default value 1.2 v

The assay we have developed requires the amplification of the entire mtDNA coding region. To reduce the time and cost of the assay we developed a multiplex amplification reaction that combines fragments 1, 3, 5, and 7 into one multiplex amplification and fragments 2, 4, and 6 into a second. This allows all of the necessary mtDNA to be amplified in just two wells before combining the reaction for downstream processing.

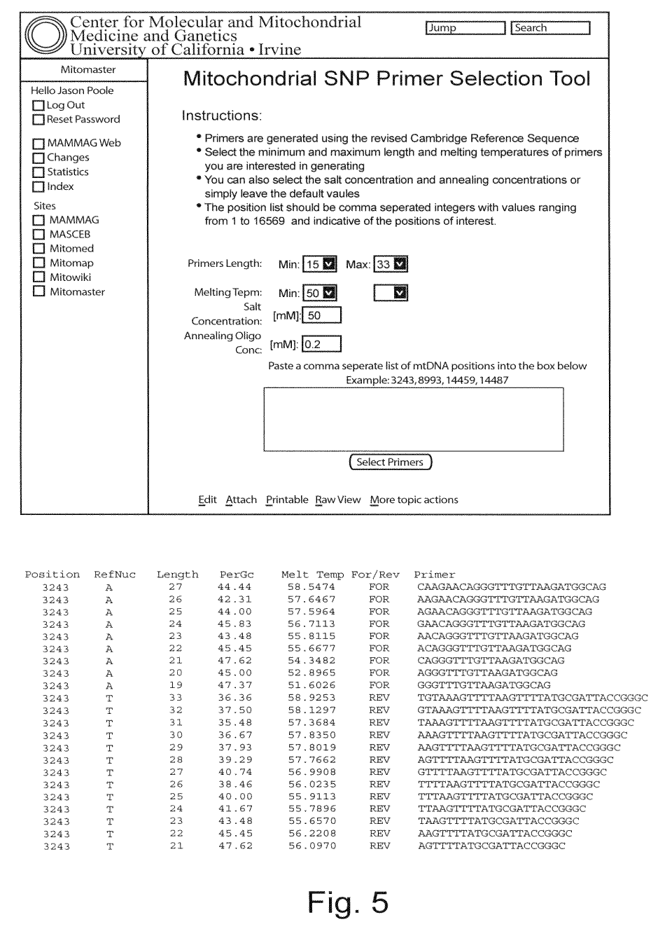

To provide a preliminary list of multiplex capable primers containing the appropriate length and melting temperatures, we have written and implemented an automated primer selection program taking into account the length, melting temperature, and SNP site of interest. The program first reads in a user provided list of SNP sites. It then queries our mtDNA database and returns a series of putative primers based on the queried mtDNA nucleotide positions. Next a series of properties about those primers are calculated including the reference nucleotide at that location (RefNuc), the primer length (Length), its melting temperature (MeltTemp), whether it was generated from the forward or reverse strand (For/Rev), and the primer sequence (Primer Sequence). Finally the data is output in a tabular format where the user can perform the final primer selection based on her criteria. The program calculates and prints both upstream and downstream primer sequences based on the user provided SNP list in the standard 5′ to 3′ format used for ordering primers. The user may also specify how many upstream and downstream primers they wish the program to generate. For example specifying the nucleotide 3010 and inputting a minimum of 15 and maximum of 33 into the program options will provide the forward and reverse primers from 15 to 33 nt in length. In addition the program allows the user to limit the primer output to only those falling within a user specified melting temperature. This is a useful feature for pre-screening primers, which do not fall within the basic melting temperature guidelines of the user's assay. A program output generated by the primer generation program is depicted in FIG. 5.

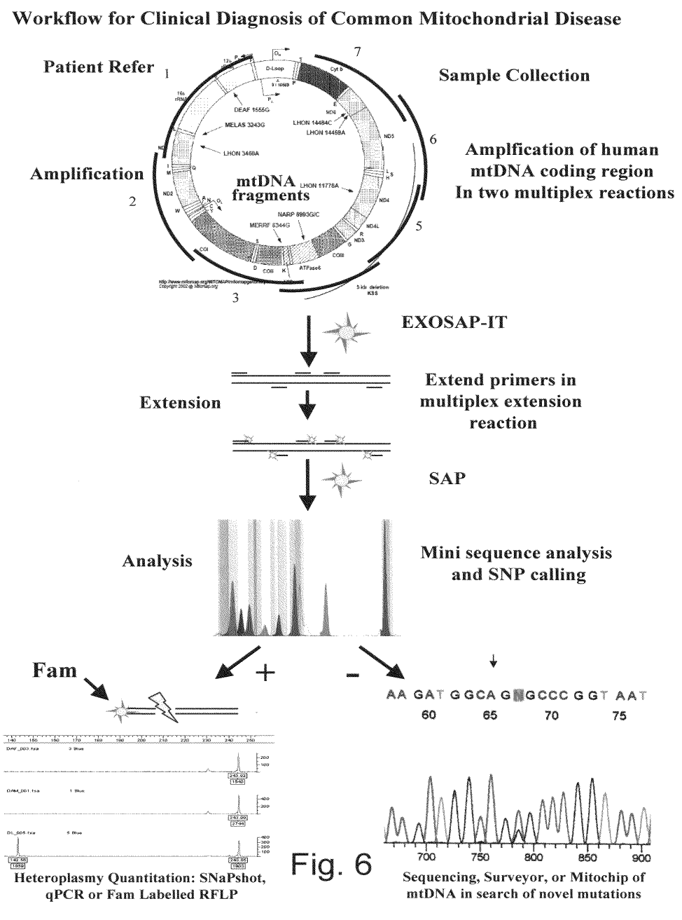

FIG. 6 provides a flowchart that schematically depicts the steps of the method described in this Example.

In both mitochondrial and nuclear based diseases it is increasingly clear that an individual's personal genetic background can either predispose or protect them from being adversely affected by a pathogenic polymorphism. Thus for most diseases, a given individuals risk is not present or absent, but found somewhere along a Poisson distribution. This same principle allows certain individuals with the same disease to respond favorably to certain drugs or treatments whereas others see little or no positive benefit. Among these findings are diseases such as Alzheimer, Parkinsons, and Prostate Cancer along with a host of other diseases (Ross, et al., 2003). For example, Leber's Hereditary Optic Neuropathy is over-represented in haplogroups J and T (Sadun, et al., 2004). Coronary artery Disease is also over represented in haplogroup T (UMDF mtg: Austrian group; sepsis Hg:H protective; AIDs hg:J—faster progression). In contrast to these findings haplogroup J was found to be enriched in centenarians and therefore protective against the diseases of aging. Oddly enough these seemingly contradictory findings are proposed to occur through the same general mechanism, a partially uncoupled electron transport chain that will be deficient in energy production, creating neuronal deficiency and death in Leber's patients but also less reactive oxygen species and therefore less cellular damage and aging in centenarians. By providing a low cost, high throughput screen that can easily be focused onto a specific population, it may be possible to further isolate haplogroup specific disease tendencies.

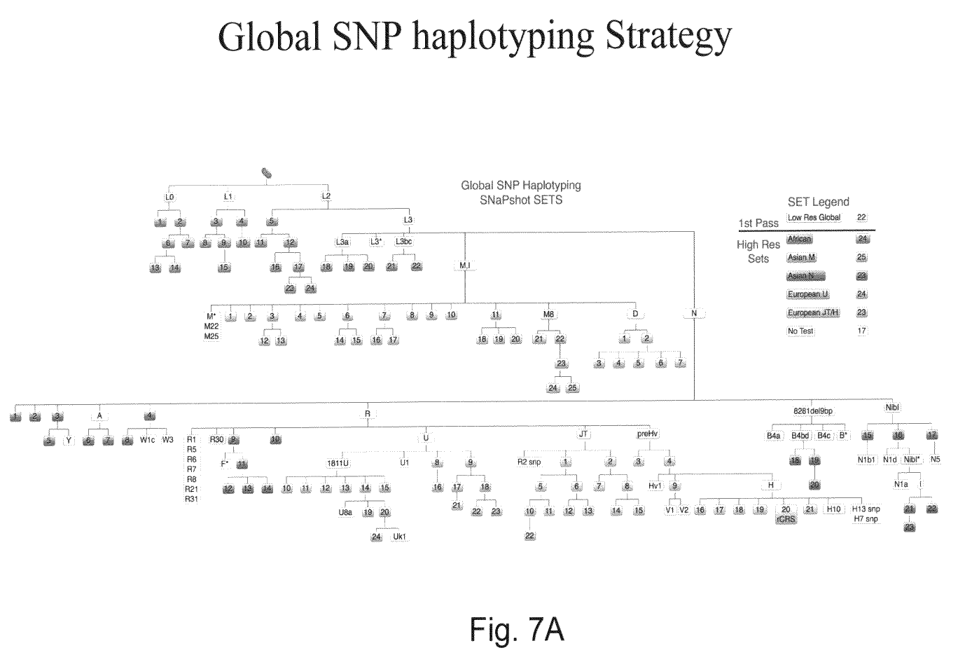

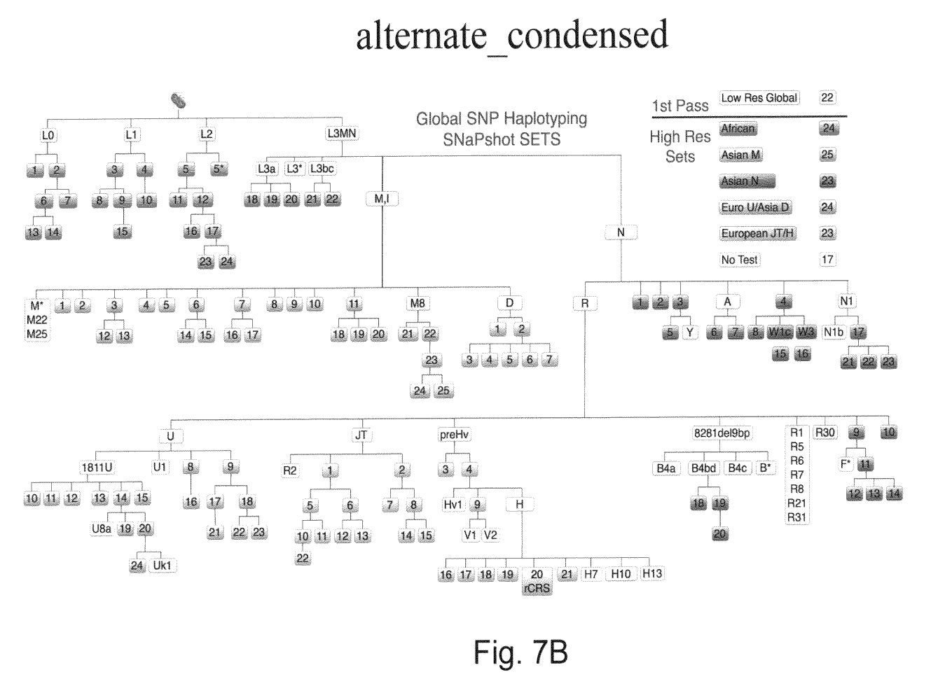

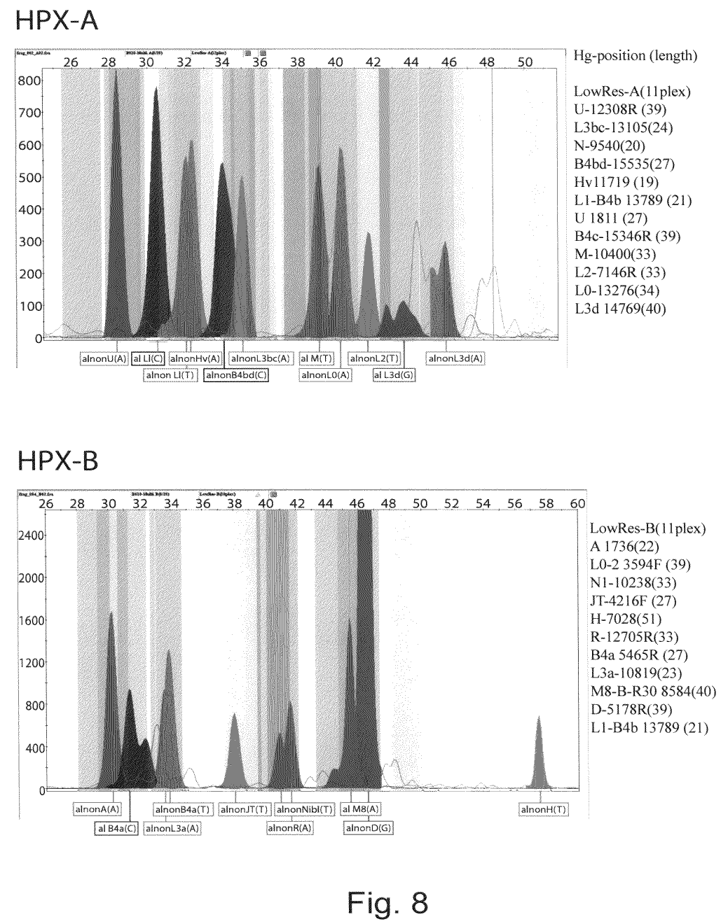

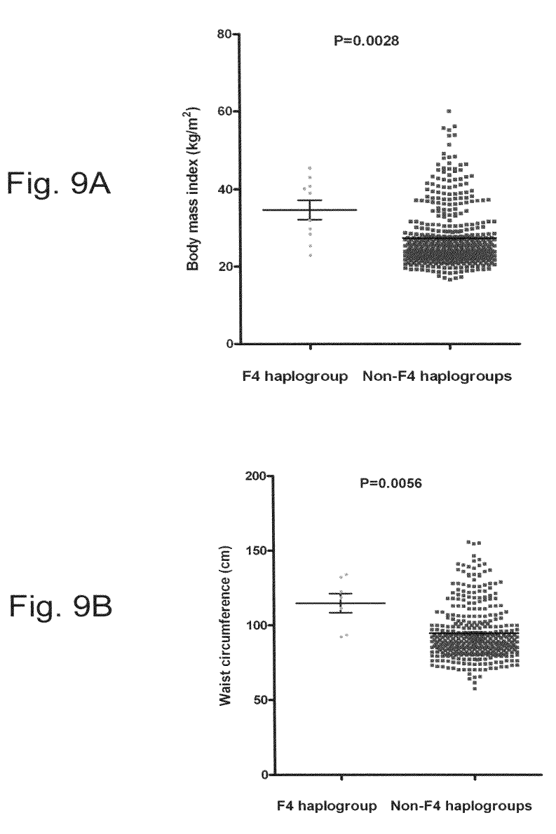

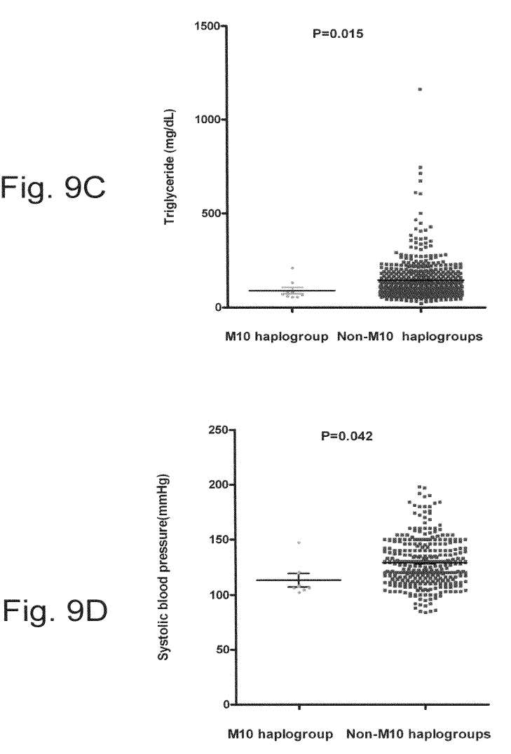

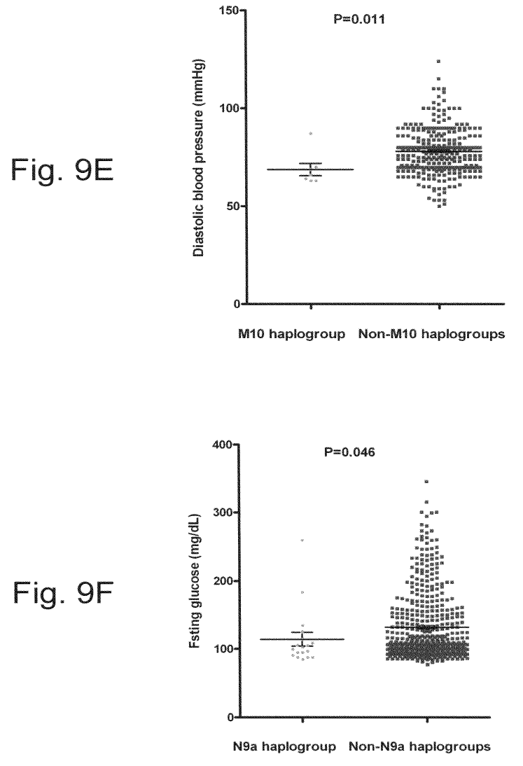

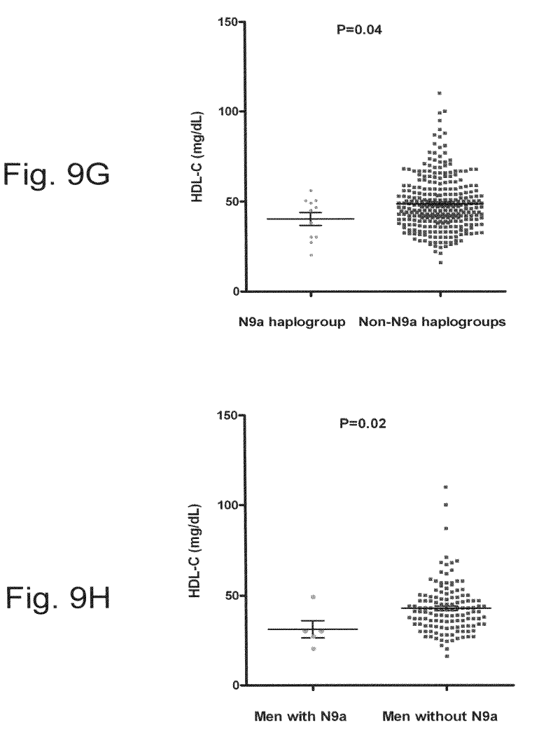

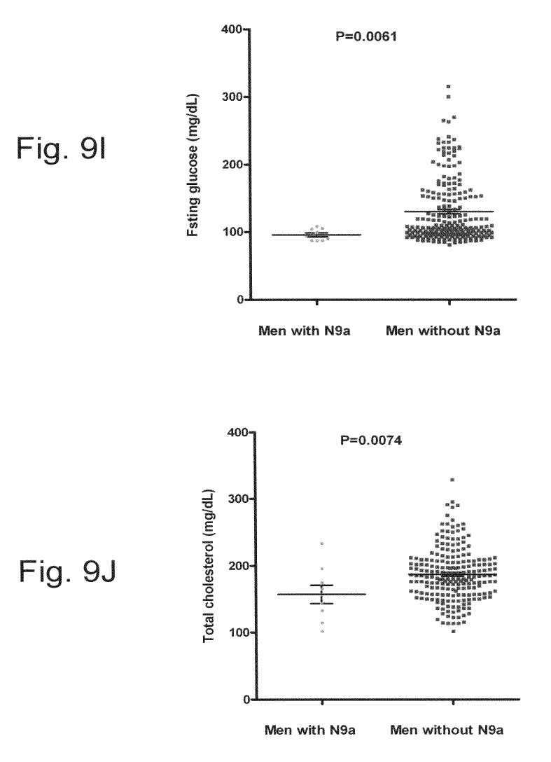

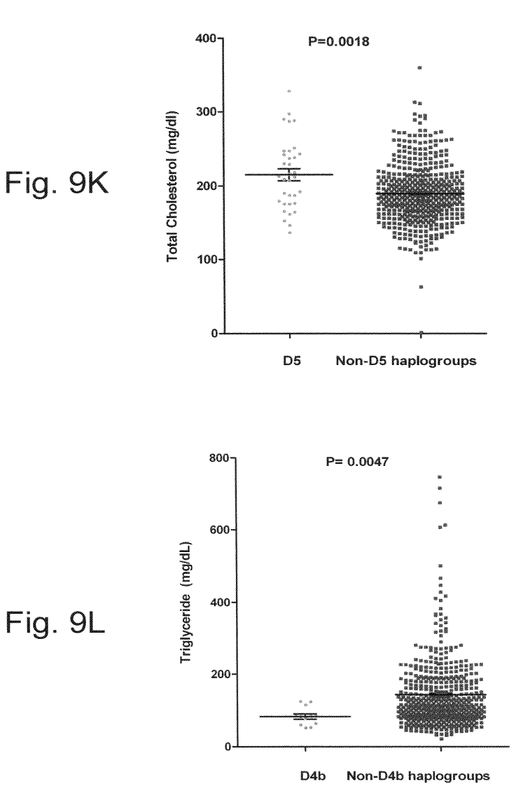

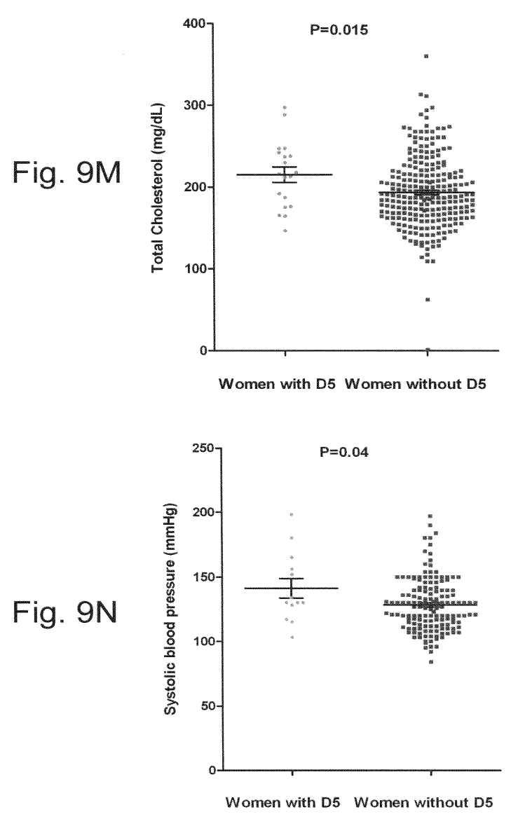

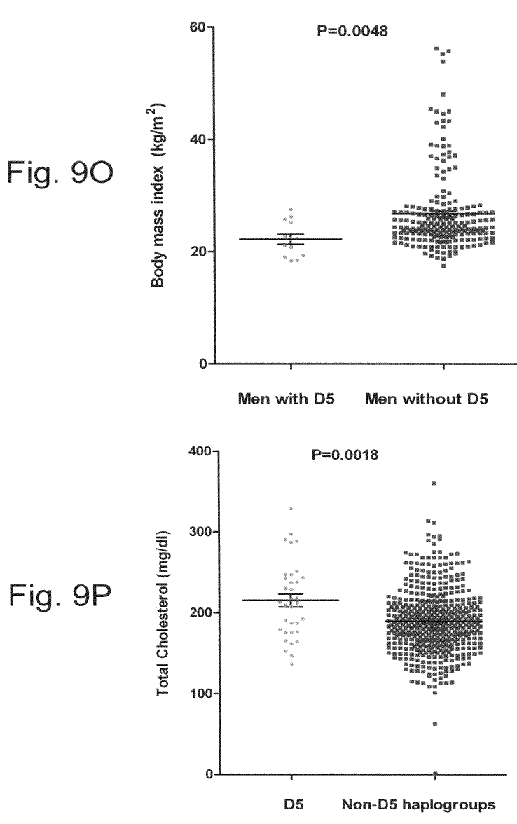

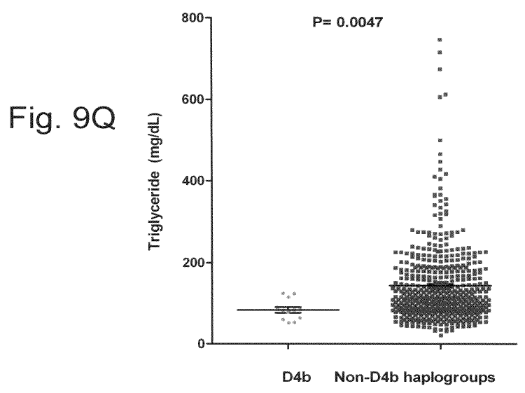

Therefore, we have devised a hierarchical strategy for identifying the mtDNA haplogroups of an unknown sample based on the information in our database of 2452 complete sequences (FIG. 7) (Ruiz-Pesini, et al., 2007).

In this test, the entire human mtDNA phylogenic tree can be subdivided using 151 highly conserved polymorphic sites located at key haplogroup branch points. To minimize unnecessary testing, we have taken advantage of the sequential mutational character of the mtDNA tree to create a sequential analysis of unknown samples. Initially the samples are tested for 22 primary sites multiplexed in two panels (internal yellow nodes in FIG. 7), which effectively subdivide the tree into major regional haplogroup units. Depending on the subdivision obtained in the first screen, each sample can be further subdivided by analysis of a subcluster of informative SNP sites. Hence samples can be defined to a high degree using the minimal number of tests. This technique can be used to complement HVI/II sequencing, and is less prone to mischaracterization due to its built in redundancy and avoidance of hypermutable sites found in the control region (Coskun, et al., 2003).

We have presented a diagnostic assay detecting 23 of the most common pathogenic mtDNA mutations and 3 disease associated haplotype positions. We envision this primer extension test as a logical first step in a work flow designed to diagnose or rule out the mtDNA as causal for mitochondrial disease. Advances in the diagnosis of mitochondrial disease have increased the accuracy and incidence of their diagnosis in recent years, but until now a complete strategy for mitochondrial disease diagnosis has not existed. This is due to the fact that heteroplasmy and mutational load can cause phenotypic changes in the clinical presentation of the disease and a combinatorial effect of several mutations from both the nuclear and mitochondrial genomes may in fact contribute to the pathology. For example the 3243 mutation may contribute to diabetes in patients with heteroplasmy as low as 1 percent but can cause seizures and stroke-like episodes when present at levels over 50 percent. Because this level of heteroplasmy can be critical in a clinical diagnosis, accurate measurements of the level of mutation can be crucial to a proper diagnosis and can have important implications for treatment options. Our strategy is capable of detecting this level of heteroplasmy (see FIG. 2) and should help clinicians and clinical investigators diagnose or rule out mitochondrial DNA as a cause of patient illness.

There are currently certain limited caveats that should be made when making a determination to exclude mtDNA from the etiology of disease. These are cases in which there is a complex genetic basis for the disease. In such cases mitochondria may play a secondary or co-dependent role in the pathogenesis of the illness and a particular mitochondrial background will influence disease progression or pathogenicity but may be dependent on a specific nuclear background. At the moment there are few examples of this as they are difficult to diagnose.

For disease diagnosis mispriming at nuclear pseudogene sites is an important consideration. Mutational load in mitochondrial disorders can affect the phenotypic expression of the disease and lead to misinterpretation of results. In some cases mtDNA pseudogene amplification could show up as sample heteroplasmy when in fact the offending polymorphism resides in the nucleus. Alternatively, if the nuclear product is preferentially amplified, the incorrect polymorphism could be detected in downstream applications. These circumstances can further complicate an already complex diagnosis and could potentially cause a clinician to prescribe an unnecessary treatment or misdiagnose the patient. To avoid all nuclear pseudogene amplification, our primer sets were tested with DNA isolated from a rho zero cell line lacking mitochondrial DNA during development.

Unequal amplification efficiency of PCR products is also a problem that can be caused by secondary structure of DNA that most often occurs in regions coding for tRNA which carry a strong folding tendency. Mutations in these areas can affect the read-through capabilities and fidelity of a polymerase adversely affecting the endogenous wildtype to mutant mtDNA ratios. This problem may arise during the PCR amplification step though unequal amplification or during primer extension. Through nucleotide misincorporation. (This could easily be tested using the MT and WT plasmids from the clinical lab and real-time PCR).

### FIGURE LEGENDS

FIG. 1 Control column shows the peak distribution present in a healthy individual. For each individual graph, length in base pairs is shown along the relative fluorescence is shown along the Rows indicate separate panels for either the multiple disease test (MD1 and MD2) or the LHON panels (LD1 and LD2). Peak positions represent the following polymorphisms. MD1: 1a-3460 (LHON); 2-3271 (MELAS); 3-1555 (DEAF) 4-14459 (LHON); 5-8344 (MERFF); 6-11778 (LHON); 7-7445 (DEAF/SNHL) MD2: 1-14484 (LHON); 2-3243 (MELAS) 3-8993 (NARP/LS-NARP 4A-14487 (LEIGHS) 5-8356 (MERRF) 6-10191 (LEIGHS) LD1: 1-3460 (LHON) 2-3733 (LHON) 3-4a-5-6-7-8-

FIG. 2 Calibration curves of mtDNA extension peak heights using previously quantified and normalized PCR products. A.) Calibration curve of mutant mtDNA. B.) Calibration curve of wild-type mtDNA.

FIG. 3. Correlation of expected and observed percentages of 3243 A→G heteroplasmy by plotting against the 3243 Standard curves from DNA samples taken from previously characterized 143B mutant at the 3243 position.

FIG. 4: Extension directly from non-amplified DNA. Patterns are identical to amplified DNA, but peaks are weaker, but non-specific peaks are eliminated.

FIG. 5: Showing the front-end form for the primer selection program used to generate extension primers in this paper

FIG. 6: Workflow for diagnosing mitochondrial disease. The entire coding region of patient DNA received by the clinical lab is amplified using multiplex amplification. A portion is EXOSAP treated for use in primer extension and the rest is set aside for possible use in sequencing downstream sequencing analysis reactions. After extension and analysis samples with verified mutations are sent for heteroplasmy quantification if necessary. Samples that show no mutation are processed further through sequencing, surveyor or mitochip analysis in search of novel mtDNA mutations.

FIG. 7. Organization of the Global Haplotyping Tree. Sections for Haplotyping are organized into color-coded sections based on the set they are assayed with. Positions are indicated below showing the haplogroup, position and haplogroup specific nucleotide at that location:

### Low Resolution Global SNPs (Yellow):

L0(9347G+13276G), L1(7055G+7389C+13789C), L2(7146G), L3(769G+1018G+3594C), L3a(10189G), L3*(14769G), L3bc(13105G), M(10400T), I(15043A), M8(8584A), D(5178A), N(9540T+10398A+10873T), Y(10398G), A(1736G), R(12705C), R30(8584A), U(12308G), U1811(1811G), Uk1(10398G), JT(4216C), preHv(11719G), H(7028C), H10(4216C), B4a(5465C), B4bd(15535T), B4c(15346C), B*(8584A), NiB1(10238C), NibI*(10398G+15043A).

### African SNPs (Green):

1(4232T) 2(9818G), 3(8027G), 4(5046C), 5(8206G), 5(13590G), 6(9755G+15431G), 7(7257A), 8(6917G), 9(10321T), 10(3693G), 11(5069(T)), 12(11944T), 13(5096A), 15(6150G), 16(7771A), 17(7624(A)), 18(14905C), 19(9554G), 20+22(6221A), 14+21(5147G), 23(4767A), 24(15849C).

### Asian D/European U SNPs (Purple):

1(5301T), 2(3010G), 2(8414G), 3(2092C), 4(3316G), 5(14979T), 6(9824A), 7(10181C), 8(3348T), 9(9477G), 10(13020A), 11(15454A), 12(5360C), 13(15693T), 14(9698T), 15(6386C), 16(7805C), 17(14793A), 18(14182A), 19(14167C), 20(14798A), 21(15218G), 22(13637A), 23(5656T), 24(9716T).

### Asian M (Orange)

1(12403G), 2(15431C), 3(4491C), 4(15218A), 5(8108), 6(4833A), 7(12940G), 8(10986), 9(5319T), 10(11482), 11(9824), 12(7598G), 13(3394T), 14(15497G), 15(13563T), 16(14025A), 17(11061C), 18(4958A), 19(7853G), 20(5442T), 21(9090A), 22(14318T), 23(15204A), 24(12672A), 25(3816T).

### Asian N (Dark Blue):

1(8404A), 2(9140C), 3(5417G), 4(5460G+8994G), 5(12358T), 6(8563T), 7(8027G), 8(9612G), 9(3970G+13928C), 10(10118A), 11(10310C), 12(10609T), 13(12338T+13708G), 14(5263G), 15(9335G), 16(13780A), 17(6221T+14470T), 18(11914C), 19(4820G), 20(9950T), 21(6734C), 22(15758A), 23(8616(T)).

### European JT/H (Light Blue):

1(13708G), 2(4917A), 3(13188C), 4(14766C), 5+16(3010C), 6(15257G), 7(12633C), 8(14233A), 9(4580G), 10(14798T), 11(5460G+13879A), 12(15812G), 13(10499T), 14(13965A), 15(5147G), 17(4024A+5004A), 18(4336T), 19(3915G), 20(1438A+4769T), 21(6776T), 22(3394).

### REFERENCES

Brown M D, Sun F, Wallace D C. 1997. Clustering of Caucasian Leber hereditary optic neuropathy patients containing the 11778 or 14484 mutations on an mtDNA lineage. Am J Hum Genet 60 (2):381-7.
Brown M D, Voljavec A S, Lott M T, MacDonald I, Wallace D C. 1992a. Leber's hereditary optic neuropathy: a model for mitochondrial neurodegenerative diseases. Faseb J 6 (10):2791-9.
Brown M D, Voljavec A S, Lott M T, Torroni A, Yang C C, Wallace D C. 1992b. Mitochondrial DNA complex I and III mutations associated with Leber's hereditary optic neuropathy. Genetics 130 (1):163-73.
Brown M D, Yang C C, Trounce I, Torroni A, Lott M T, Wallace D C. 1992c. A mitochondrial DNA variant, identified in Leber hereditary optic neuropathy patients, which extends the amino acid sequence of cytochrome c oxidase subunit I. Am J Hum Genet 51 (2):378-85.
Cassandrini D, Calevo M G, Tessa A, Manfredi G, Fattori F, Meschini M C, Carrozzo R, Tonoli E, Pedemonte M, Minetti C and others. 2006. A new method for analysis of mitochondrial DNA point mutations and assess levels of heteroplasmy. Biochem Biophys Res Commun 342 (2):387-93.
Chomyn A, Martinuzzi A, Yoneda M, Daga A, Hurko O, Johns D, Lai S T, Nonaka I, Angelini C, Attardi G. 1992. MELAS mutation in mtDNA binding site for transcription termination factor causes defects in protein synthesis and in respiration but no change in levels of upstream and downstream mature transcripts. Proc Natl Acad Sci USA 89 (10):4221-5.
Coskun P E, Ruiz-Pesini E, Wallace D C. 2003. Control region mtDNA variants: longevity, climatic adaptation, and a forensic conundrum. Proc Natl Acad Sci USA 100 (5):2174-6.
Genasetti A, Valentino M L, Carelli V, Vigetti D, Viola M, Karousou E G, Melzi d'Eril G V, De Luca G, Passi A, Pallotti F. 2007. Assessing heteroplasmic load in Leber's hereditary optic neuropathy mutation 3460G->A/MT-ND1 with a real-time PCR quantitative approach. J Mol Diagn 9 (4):538-45.
Goto Y, Nonaka I, Horai S. 1991. A new mtDNA mutation associated with mitochondrial myopathy, encephalopathy, lactic acidosis and stroke-like episodes (MELAS). Biochim Biophys Acta 1097 (3):238-40.
Holt I J, Harding A E, Petty R K, Morgan-Hughes J A. 1990. A new mitochondrial disease associated with mitochondrial DNA heteroplasmy. Am J Hum Genet 46 (3):428-33.
Luberichs J, Leo-Kottler B, Besch D, Fauser S. 2002. A mutational hot spot in the mitochondrial ND6 gene in patients with Leber's hereditary optic neuropathy. Graefes Arch Clin Exp Opthalmol 240 (2):96-100.
Procaccio V, Neckelmann N, Paquis-Flucklinger V, Bannwarth S, Jimenez R, Davila A, Poole J C, Wallace D C. 2006. Detection of low levels of the mitochondrial tRNALeu(UUR) 3243A>G mutation in blood derived from patients with diabetes. Mol Diagn Ther 10 (6):381-9.
Reid F M, Vernham G A, Jacobs H T. 1994. A novel mitochondrial point mutation in a maternal pedigree with sensorineural deafness. Hum Mutat 3 (3):243-7.
Ross O A, McCormack R, Maxwell L D, Duguid R A, Quinn D J, Barnett Y A, Rea I M, El-Agnaf O M, Gibson J M, Wallace A and others. 2003. mt4216C variant in linkage with the mtDNA TJ cluster may confer a susceptibility to mitochondrial dysfunction resulting in an increased risk of Parkinson's disease in the Irish. Exp Gerontol 38 (4):397-405.
Ruiz-Pesini E, Lott M T, Procaccio V, Poole J C, Brandon M C, Mishmar D, Yi C, Kreuziger J, Baldi P, Wallace D C. 2007. An enhanced MITOMAP with a global mtDNA mutational phylogeny. Nucleic Acids Res 35 (Database issue):D823-8.
Sadun F, De Negri A M, Carelli V, Salomao S R, Berezovsky A, Andrade R, Moraes M, Passos A, Belfort R, da Rosa A B and others. 2004. Opthalmologic findings in a large pedigree of 11778/Haplogroup J Leber hereditary optic neuropathy. Am J Opthalmol 137 (2):271-7.
Tanaka M, Ino H, Ohno K, Ohbayashi T, Ikebe S, Sano T, Ichiki T, Kobayashi M, Wada Y, Ozawa T. 1991. Mitochondrial DNA mutations in mitochondrial myopathy, encephalopathy, lactic acidosis, and stroke-like episodes (MELAS). Biochem Biophys Res Commun 174 (2):861-8.
Valentino M L, Avoni P, Barboni P, Pallotti F, Rengo C, Torroni A, Bellan M, Baruzzi A, Carelli V. 2002. Mitochondrial DNA nucleotide changes C14482G and C14482A in the ND6 gene are pathogenic for Leber's hereditary optic neuropathy. Ann Neurol 51 (6):774-8.
Vallone P M, Just R S, Coble M D, Butler J M, Parsons T J. 2004. A multiplex allele-specific primer extension assay for forensically informative SNPs distributed throughout the mitochondrial genome. Int J Legal Med 118 (3):147-57.
Wallace D C. 2005. A mitochondrial paradigm of metabolic and degenerative diseases, aging, and cancer: a dawn for evolutionary medicine. Annu Rev Genet 39:359-407.
Wallace D C, Lott M T, Shoffner J M, Brown M D. 1992. Diseases resulting from mitochondrial DNA point mutations. J Inherit Metab Dis 15 (4):472-9.

It is understood that the examples and embodiments described herein are for illustrative purposes only and that various modifications or changes in light thereof will be suggested to persons skilled in the art and are to be included within the spirit and purview of this application and scope of the appended claims.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed is:

1. A method of identifying a metabolic syndrome phenotype for an organism or biological sample derived therefrom, the method comprising:
detecting, in the organism or biological sample, a polymorphism, haplotype, haplotype subgroup or haplotype group in a mitochondrial genome of the organism; and,
correlating the polymorphism or haplotype to the metabolic syndrome phenotype.

2. A method of identifying a metabolic syndrome phenotype for an organism or biological sample derived therefrom, the method comprising:
detecting, in the organism or biological sample, a polymorphism, haplotype, haplotype subgroup or haplotype group noted in the tables herein, wherein the polymorphism, haplotype, haplotype subgroup or haplotype group is associated with the metabolic syndrome phenotype; and,
correlating the polymorphism, haplotype, haplotype subgroup or haplotype group to the metabolic syndrome phenotype.

3. The method of claim 1 or 2, wherein the correlation between a metabolic syndrome phenotype and a haplotype or haplotype subgroup comprises one or more of: E and waist circumference; F3 and waist circumference; F4 and increased risk for obesity, waist circumference and body mass index (BMI); M10 and decreased levels of triglycerides and systolic and diastolic blood pressure (SBP, DBP); N9a and decreased risk for type 2 diabetes (T2DM), cholesterol, and high density lipoprotein levels (HDL) in men; R9 and overall metabolic syndrome (MS); D and increased SBP; D5 and elevated cholesterol and SBP in women but decreased BMI for men; and D4b for very low triglycerides.

4. The method of claim 1 or 2, wherein the organism is a human patient, or the biological sample is derived from a human patient.

5. The method of claim 1 or 2, wherein the detecting comprises amplifying the polymorphism or a sequence associated therewith and detecting the resulting amplicon.

6. The method of claim 5, wherein the amplicon is detected by a process that includes one or more of: hybridizing the amplicon to an array, digesting the amplicon with a restriction enzyme, or real-time PCR analysis.

7. The method of claim 5, comprising partially or fully sequencing the amplicon.

8. The method of claim 5, wherein the amplifying comprises performing a polymerase chain reaction (PCR), reverse transcriptase PCR (RT-PCR), or ligase chain reaction (LCR) using nucleic acid isolated from the organism or biological sample as a template in the PCR, RT-PCR, or LCR.

9. The method of claim 1 or 2, wherein correlating the polymorphism comprises referencing a look up table that comprises correlations between alleles of the polymorphism and the phenotype.

10. A system or kit comprising:
an amplification composition or set of amplification reagents comprising one or more amplification primers that flank or comprise one or more polymorphisms that distinguish one or more haplotypes, haplotype subgroup or haplotype groups selected from: E, F3, F4, M10, N9a, R9, D, D5 and D4b; and,
a look up table that correlates one or more of the haplotypes, haplotype subgroups or haplotype groups to one or more metabolic syndrome phenotype.

11. The system or kit of claim 10, comprising one or more containers that contain the amplification composition or amplification primers.

12. The system or kit of claim 10, comprising computer-implemented instructions that correlate a product of the amplification composition or reagents with the metabolic syndrome phenotype using the look up table.

13. A method of identifying a modulator of a metabolic syndrome phenotype, the method comprising:
contacting a potential modulator to a gene or gene product, wherein the gene or gene product comprises a polymorphism within, or is at least partially encoded within a haplotype selected from: E, F3, F4, M10, N9a, R9, D, D5 and D4b; and,
detecting an effect of the potential modulator on the gene or gene product, thereby identifying whether the potential modulator modulates the metabolic syndrome phenotype.

14. The method of claim 13, wherein the metabolic syndrome phenotype comprises insulin resistance, a lipid disorder, or central obesity.

15. The method of claim 13, wherein the effect comprises increased or decreased expression of a gene encoding or corresponding to a polymorphism or haplotype herein in the presence of the modulator.

16. A kit for treatment of a metabolic syndrome phenotype, the kit comprising a modulator identified by the method of claim 13 and instructions for administering the compound to a patient to treat the metabolic syndrome phenotype.

17. The kit of claim 16, wherein the metabolic syndrome phenotype is an obesity predisposition, dyslipidemia, or an insulin resistance phenotype.


SEQUENCE LISTING (22 sequences)

SEQ ID NO: 1 - DNA, 27 bp, Artificial Sequence
  Oligonucleotide
caagaacagggtttgttaagatggcag

SEQ ID NO: 2 - DNA, 26 bp, Artificial Sequence
  Oligonucleotide
aagaacagggtttgttaagatggcag

SEQ ID NO: 3 - DNA, 25 bp, Artificial Sequence
  Oligonucleotide
agaacagggtttgttaagatggcag

SEQ ID NO: 4 - DNA, 24 bp, Artificial Sequence
  Oligonucleotide
gaacagggtttgttaagatggcag

SEQ ID NO: 5 - DNA, 23 bp, Artificial Sequence
  Oligonucleotide
aacagggtttgttaagatggcag

SEQ ID NO: 6 - DNA, 22 bp, Artificial Sequence
  Oligonucleotide
acagggtttgttaagatggcag

SEQ ID NO: 7 - DNA, 21 bp, Artificial Sequence
  Oligonucleotide
cagggtttgttaagatggcag

SEQ ID NO: 8 - DNA, 20 bp, Artificial Sequence
  Oligonucleotide
agggtttgttaagatggcag

SEQ ID NO: 9 - DNA, 19 bp, Artificial Sequence
  Oligonucleotide
gggtttgttaagatggcag

SEQ ID NO: 10 - DNA, 33 bp, Artificial Sequence
  Oligonucleotide
tgtaaagttttaagttttatgcgattaccgggc

SEQ ID NO: 11 - DNA, 32 bp, Artificial Sequence
  Oligonucleotide
gtaaagttttaagttttatgcgattaccgggc

SEQ ID NO: 12 - DNA, 31 bp, Artificial Sequence
  Oligonucleotide
taaagttttaagttttatgcgattaccgggc

SEQ ID NO: 13 - DNA, 30 bp, Artificial Sequence
  Oligonucleotide
aaagttttaagttttatgcgattaccgggc

SEQ ID NO: 14 - DNA, 29 bp, Artificial Sequence
  Oligonucleotide
aagttttaagttttatgcgattaccgggc

SEQ ID NO: 15 - DNA, 28 bp, Artificial Sequence
  Oligonucleotide
agttttaagttttatgcgattaccgggc

SEQ ID NO: 16 - DNA, 27 bp, Artificial Sequence
  Oligonucleotide
gttttaagttttatgcgattaccgggc

SEQ ID NO: 17 - DNA, 26 bp, Artificial Sequence
  Oligonucleotide
ttttaagttttatgcgattaccgggc

SEQ ID NO: 18 - DNA, 25 bp, Artificial Sequence
  Oligonucleotide
tttaagttttatgcgattaccgggc

SEQ ID NO: 19 - DNA, 24 bp, Artificial Sequence
  Oligonucleotide
ttaagttttatgcgattaccgggc

SEQ ID NO: 20 - DNA, 23 bp, Artificial Sequence
  Oligonucleotide
taagttttatgcgattaccgggc

SEQ ID NO: 21 - DNA, 22 bp, Artificial Sequence
  Oligonucleotide
aagttttatgcgattaccgggc

SEQ ID NO: 22 - DNA, 21 bp, Artificial Sequence
  Oligonucleotide
agttttatgcgattaccgggc